(19) United States
(12) Patent Application Publication
(10) Pub. No.: US 2021/0398304 A1
(43) Pub. Date: Dec. 23, 2021

(54) MEDICAL OBSERVATION SYSTEM CONFIGURED TO GENERATE THREE-DIMENSIONAL INFORMATION AND TO CALCULATE AN ESTIMATED REGION AND A CORRESPONDING METHOD

(71) Applicant: SONY CORPORATION, Tokyo (JP)

(72) Inventors: Keisuke UYAMA, Tokyo (JP); Tsuneo HAYASHI, Tokyo (JP)

(73) Assignee: Sony Group Corporation, Tokyo (JP)

(21) Appl. No.: 17/283,962

(22) PCT Filed: Nov. 7, 2019
(86) PCT No.: PCT/JP2019/043657

§ 371 Date: Apr. 9, 2021

(30) Foreign Application Priority Data
Nov. 7, 2018 (JP) 2018-210100

(51) Int. Cl. G06T 7/55; A61B 1/04; A61B 1/06; G06T 5/00

(52) U.S. Cl. CPC G06T 7/55; A61B 1/043; A61B 1/0638; G06T 2207/10068; G06T 2207/10064; G06T 2207/30004; G06T 5/001

(57) ABSTRACT

A medical observation system (1000), a signal processing apparatus and a medical observation method are provided to suppress effects of variations with time in an observation target. The medical observation system includes a generating section (21) that generates three-dimensional information regarding an operative field, a setting section (31) that sets, based on a special light image captured by a medical observation apparatus during illumination of special light having a predetermined wavelength bandwidth, an interested region in the special light image, a calculating section (32) that estimates, from the three-dimensional information, an estimated region corresponding to a physical position of the interested region in a normal light image captured by the medical observation apparatus during illumination of normal light having a wavelength bandwidth different from the predetermined wavelength bandwidth, and an image processing section (41) that applies predetermined image processing to the estimated region in the normal light image.

### TECHNICAL FIELD

The present disclosure relates to a medical observation system, signal processing apparatus, and medical observation method.

### BACKGROUND ART

In surgery using an endoscope, there is an increasing trend that normal light observation, in which observation of an operative field is performed by illuminating normal illumination light (for example, white light), and special light observation, in which observation of an operative field is performed by illuminating special light of a wavelength bandwidth different from that of normal illumination light, are differently used depending on the operative field.

In special light observation, a biomarker such as, for example, a phosphor is used to facilitate differentiating an observation target from other sites. Injection of the biomarker into the observation target causes the observation target to emit fluorescence, so that a surgeon and the like can easily differentiate the observation target and the other sites.

### CITATION LIST

### Patent Literature

PTL 1: JP 2012-50618A

### SUMMARY

### Technical Problem

However, a biomarker used in special light observation may undergo diffusion or quenching with time, thereby making it difficult to differentiate an observation target from other sites. In other words, variations occur with time in an observation target.

The present disclosure therefore proposes a medical observation system, signal processing apparatus and medical observation method, which can suppress effects of variations with time in an observation target.

### Solution to Problem

To resolve the above-described problem, a medical observation system of an embodiment according to the present disclosure includes: circuitry configured to obtain a first surgical image captured by a medical imaging apparatus during illumination in a first wavelength band and a second surgical image captured by the medical imaging apparatus during illumination in a second wavelength band different from the first wavelength band, generate three-dimensional information regarding an operative field, obtain information of an interested region in the first surgical image, calculate, based on the three-dimensional information, an estimated region in the second surgical image corresponding to a physical position of the interested region, and output the second surgical image processed a predetermined image processing on the estimated region.

### BRIEF DESCRIPTION OF DRAWINGS

FIG. 1 is a diagram depicting an example of a schematic configuration of an endoscopic surgery system to which techniques according to the present disclosure can be applied.

FIG. 2 is a functional block diagram depicting a functional configuration of a medical observation system.

FIG. 3 is a diagram illustrating a method that a three-dimensional information generating section generates three-dimensional map information.

FIG. 4 is a flow chart illustrating an example of a flow of processing that the medical observation system performs.

FIG. 5A depicts images of examples of captured image data.

FIG. 5B is an image depicting an example of an interested region extracted from special light image data.

FIG. 5C is an image depicting an example of display image data with annotation information superimposed on normal light image data.

FIG. 5D is an image depicting an example of display image data with the annotation information superimposed on other normal light image data.

FIG. 6 is an image depicting an example of display image data with annotation information superimposed thereon, including information regarding an interested region.

FIG. 7 is an image depicting an example of display image data with annotation information superimposed thereon corresponding to feature values of every region contained in an interested region.

FIG. 8 is an image depicting an example of display image data with annotation information representing a blood flow and superimposed thereon.

FIG. 9A is an image depicting an example of a method for specifying an interested region.

FIG. 9B is an image depicting an example of setting of an interested region.

FIG. 10 is a diagram depicting an example of a configuration of a part of a medical observation system according to a tenth embodiment.

FIG. 11 is a diagram depicting an example of a configuration of a part of a medical observation system according to an eleventh embodiment.

FIG. 12 is a diagram depicting an example of a configuration of a part of a medical observation system according to a twelfth embodiment.

FIG. 13 is a diagram depicting an example of a configuration of a part of a medical observation system according to a thirteenth embodiment.

FIG. 14 is a diagram depicting an example of a configuration of a part of a medical observation system according to a fourteenth embodiment.

FIG. 15 is a diagram depicting an example of a configuration of a part of a medical observation system according to a fifteenth embodiment.

FIG. 16 is a diagram depicting an example of a configuration of a part of a medical observation system according to a sixteenth embodiment.

FIG. 17 is a diagram depicting an example of a configuration of a part of a medical observation system according to a seventeenth embodiment.

FIG. 18 is a diagram depicting an example of a configuration of a part of a medical observation system according to an eighteenth embodiment.

FIG. 19 is a diagram depicting an example of a configuration of a part of a medical observation system according to a nineteenth embodiment.

FIG. 20 is a diagram depicting an example of a configuration of a part of a medical observation system according to a twentieth embodiment.

FIG. 21 is a view depicting an example of a schematic configuration of a microscopic surgery system to which a technique according to the present disclosure can be applied.

FIG. 22 is a view illustrating how surgery is being performed using the microscopic surgery system 5300 depicted in FIG. 21.

### DESCRIPTION OF EMBODIMENTS

Based on the drawings, embodiments according to the present disclosure will hereinafter be described in detail. It is to be noted that in the following individual embodiments, like elements are identified by like reference signs to omit overlapping descriptions.

### First Embodiment

### Configuration of Endoscopic Surgery System According to First Embodiment

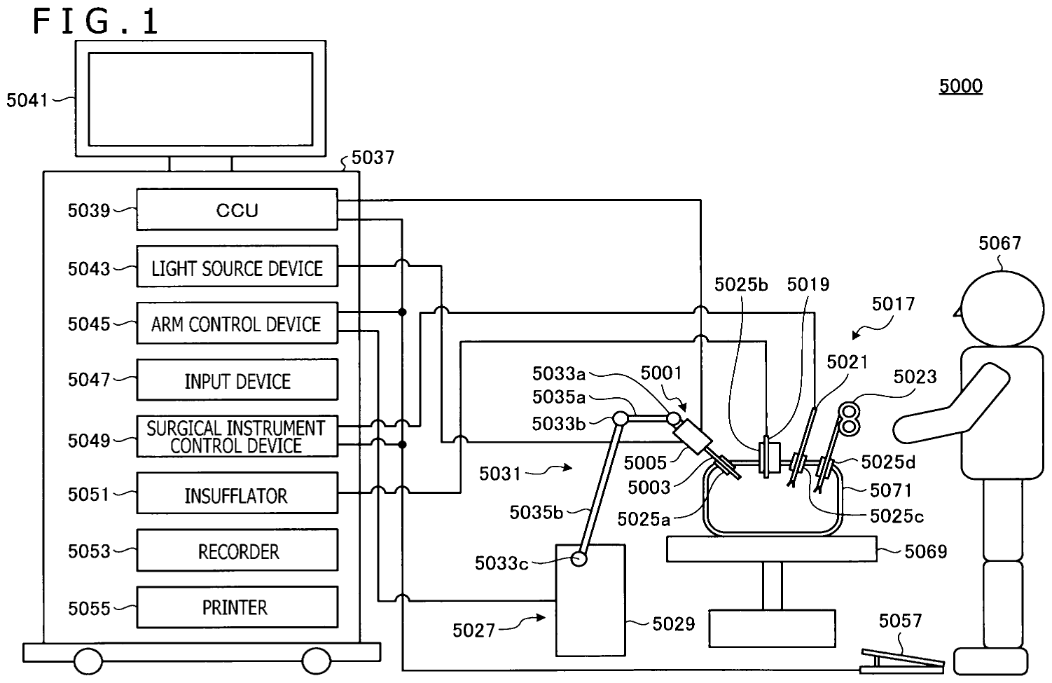

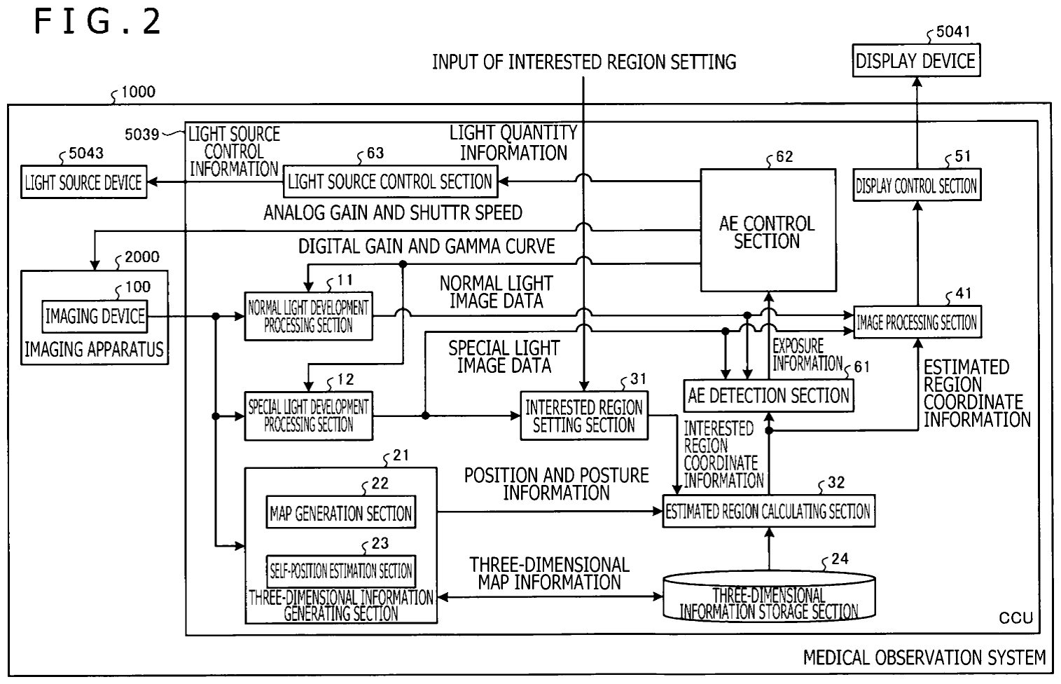

In a first embodiment, a description will be made taking as an example a case in which a medical observation system 1000 (see FIG. 2) is applied to a part of an endoscopic surgery system 5000. FIG. 1 is a diagram depicting an example of a schematic configuration of the endoscopic surgery system 5000 to which techniques according to the present disclosure can be applied. FIG. 1 depicts how an operator (surgeon) 5067 is performing surgery on a patient 5071 on a patient bed 5069 by using the endoscopic surgery system 5000. As depicted in the figure, the endoscopic surgery system 5000 is configured from an endoscope 5001 (the endoscope 5001 is an example of a medical observation apparatus), other surgical instruments 5017, a support arm device 5027 with the endoscope 5001 supported thereon, and a cart 5037 on which various devices for surgery under endoscope are mounted.

In endoscopic surgery, a plurality of cylindrical perforating tools called “trocars 5025a to 5025d” is pierced through the abdominal wall instead of incising the abdominal wall to open the abdominal cavity. Through the trocars 5025a to 5025d, a barrel 5003 of the endoscope 5001 and the other surgical instruments 5017 are then inserted into the body cavity of the patient 5071. In the depicted example, an insufflator tube 5019, an energy treatment instrument 5021 and forceps 5023 are inserted as the other surgical instruments 5017 into the body cavity of the patient 5071. Here, the energy treatment instrument 5021 is a treatment instrument that performs incision or removal of a tissue, sealing of blood vessels, or the like under high frequency electric current or ultrasonic vibrations. However, the surgical instruments 5017 depicted in the figure are merely illustrative, so that various surgical instruments commonly employed in surgery under endoscope, such as tweezers and a retractor, for example, may be used as the surgical instruments 5017.

An image of an operative field in the body cavity of the patient 5071 as captured by the endoscope 5001 is displayed on a display device 5041. The operator 5067 performs treatment such as, for example, resection of an affected area with the energy treatment instrument 5021 and forceps 5023 while watching in real time the image of the operative field displayed on the display device 5041. It is to be noted that, although depiction is omitted in the figure, the insufflator tube 5019, energy treatment instrument 5021 and forceps 5023 are supported by the operator 5067, an assistant or the like during the surgery.

(Support Arm Device)

The support arm device 5027 includes an arm portion 5031 extending from a base portion 5029. In the example depicted in the figure, the arm portion 5031 is configured from joint portions 5033a, 5033b, and 5033c and links 5035a and 5035b, and is driven under control from an arm control device 5045. By the arm portion 5031, the endoscope 5001 is supported and its position and posture are controlled. As a consequence, stable positional fixing of the endoscope 5001 can be realized.

(Endoscope)

The endoscope 5001 is configured from the barrel 5003 to be inserted over its part of a predetermined length from a distal end thereof into the body cavity of the patient 5071, a casing to which the barrel 5003 can be connected, and a camera head 5005 to be connected to a proximal end of the barrel 5003. In the example depicted in the figure, the endoscope 5001 is depicted as one configured as a so-called rigid endoscope having a rigid barrel 5003, but the endoscope 5001 may be configured as a so-called flexible endoscope having a flexible barrel 5003.

The barrel 5003 includes, at a distal end thereof, an opening with an objective lens fitted therein. To the endoscope 5001, a light source device 5043 is connected. Light generated by the light source device 5043 is guided to the distal end of the barrel through a light guide disposed extending inside the barrel 5003, and is illuminated through the objective lens toward an observation target in the body cavity of the patient 5071. It is to be noted that the endoscope 5001 may be a forward-viewing endoscope or may be a forward-oblique viewing endoscope or side-viewing endoscope.

An optical system and an imaging device are disposed inside the camera head 5005, and reflected light (observed light) from the observation target is condensed on the imaging device by the optical system. The observed light is photoelectrically converted by the imaging device, and electrical signals corresponding to the observed light, specifically image signals corresponding to an observed image are generated. The image signals are transmitted as RAW data to a camera control unit (CCU) 5039. It is to be noted that the camera head 5005 is mounted with functions to adjust the magnification and focal length by driving the optical system as needed.

It is to be noted that a plurality of imaging devices may be disposed in the camera head 5005, for example, to accommodate stereovisions (3D displays) and the like. In this case, a plurality of relay optical systems is disposed inside the barrel 5003 to guide observed light to each of the plurality of the imaging devices.

(Various Devices Mounted on Cart)

The CCU 5039 is configured by a CPU (Central Processing Unit), a GPU (Graphic Processing Unit), and the like, and comprehensively controls operations of the endoscope 5001 and display device 5041. Specifically, the CCU 5039 applies various image processing such as, for example, development processing (mosaic processing) and the like to image signals received from the camera head 5005 so that an image is displayed based on the image signals. The CCU 5039 provides the display device 5041 with the image signals subjected to the image processing. Further, the CCU 5039 transmits control signals to the camera head 5005 to control its drive. The control signals can include information regarding imaging conditions, such as a magnification, a focal length and the like. Furthermore, the CCU 5039 may be realized by an integrated circuit such as, for example, an ASIC (Application Specific Integrated Circuit) or an FPGA (Field Programmable Gate Array) without being limited to the CPU and the GPU. In other words, the functions of CCU is realized by predetermined circuitry.

Under control from the CCU 5039, the display device 5041 displays an image based on image signals subjected to image processing by the CCU 5039. In a case where the endoscope 5001 is one accommodated to imaging at high resolution such as, for example, 4K (3840 horizontal pixels×2160 vertical pixels) or 8K (7680 horizontal pixels×4320 vertical pixels), and/or in a case where endoscope 5001 is one accommodated to 3D displays, one capable of high-resolution displays and/or one capable of 3D displays can be used as the display device 5041 in correspondence to the respective cases. In the case of the one accommodated to imaging at high resolution such as 4K, 8K or the like, use of one having a size of 55 inches or greater as the display device 5041 provides a still deeper sense of immersion. In addition, a plurality of display devices 5041 of different resolutions or sizes may be disposed depending on the use.

The light source device 5043 is configured from a light source such as, for example, an LED (light emitting diode), and supplies illumination light to the endoscope 5001 upon imaging the operative field. In other words, the light source device 5043 illuminates special light, which has a predetermined wavelength bandwidth, or normal light, which has a wavelength bandwidth different from the wavelength bandwidth of the special light, to the operative field via the barrel 5003 (also called “a scope”) inserted to the operative field. In other words, the light source device includes a first light source that supplies illumination light in a first wavelength band and a second light source that supplies illumination light in a second wavelength band different from the first wavelength band. For example, the illumination light in the first wavelength band (the special light) is an infrared light (light with a wavelength of 760 nm or more), a blue light, or ultraviolet light. For example, the illumination light in the second wavelength band (the normal light) is a white light or a green light. Basically, when the special light is the infrared light or the ultraviolet light, the normal light is the white light. Further, when the special light is the blue light, the normal light is the green light.

The arm control device 5045 is configured by a processor such as, for example, a CPU, and operates according to a predetermined program, so that driving of the arm portion 5031 of the support arm device 5027 is controlled according to a predetermined control method.

An input device 5047 is an input interface for the endoscopic surgery system 5000. A user can perform an input of various kinds of information and an input of an instruction to the endoscopic surgery system 5000 via the input device 5047. For example, the user inputs various kinds of information regarding surgery, such as physical information regarding the patient and information regarding the operative method of the surgery, via the input device 5047. Further, the user also inputs, via the input device 5047, for example, an instruction to the effect that the arm portion 5031 shall be driven, instructions to the effect that conditions (the kind of illumination light, the magnification, the focal length, and the like) for imaging by the endoscope 5001 shall be changed, an instruction to the effect that the energy treatment instrument 5021 shall be driven, and so on.

No limitation is imposed on the kind of the input device 5047, and the input device 5047 may be desired one or more of various known input devices. As the input device 5047, a mouse, a keyboard, a touch panel, a switch, a foot switch 5057, a lever and/or the like can be applied, for example. In a case where a touch panel is used as the input device 5047, the touch panel may be disposed on a display screen of the display device 5041.

As an alternative, the input device 5047 is configured from devices fitted to the user, such as an eyeglass-type wearable device and an HMD (Head Mounted Display), and various inputs are performed according to gestures and sightlines of the user as detected by these devices. Further, the input device 5047 includes a camera capable of detecting movements of the user, and according to gestures and sightlines of the user as detected from images captured by the camera, various inputs are performed. Furthermore, the input device 5047 includes a microphone capable of picking up the user's voice, so that various inputs are performed by voice via the microphone. By configuring the input device 5047 to be able to input various kinds of information without contact as described above, the user (for example, the operator 5067) who belongs to a clean area can operate equipment, which belongs to an unclean area, without contact. In addition, the user can operate equipment without releasing the user's hold on a surgical instrument, and therefore the user's convenience is improved.

A surgical instrument control device 5049 controls the driving of the energy treatment instrument 5021 for cauterization or incision of a tissue, or sealing of blood vessels, or the like. To inflate the body cavity of the patient 5071 for the purpose of ensuring a field of vision for the endoscope 5001 and a working space for the operator, an insufflator 5051 supplies gas into the body cavity via the insufflator tube 5019. A recorder 5053 is a device that can record various kinds of information regarding surgery. A printer 5055 is a device that can print various kinds of information regarding surgery in various forms such as texts, images or graphs.

A description will hereinafter be made in further detail about particularly characteristic configurations in the endoscopic surgery system 5000.

(Support Arm Device)

The support arm device 5027 includes the base portion 5029 as a support, and the arm portion 5031 extending from the base portion 5029. In the example depicted in the figure, the arm portion 5031 is configured from the joint portions 5033a, 5033b, and 5033c and the links 5035a and 5035b connected together by the joint portion 5033b. In FIG. 1, the configuration of the arm portion 5031 is depicted in a simplified form for the sake of simplification. Actually, however, the shapes, number and arrangement of the joint portions 5033a to 5033c and the links 5035a and 5035b, the directions of rotational axes of the joint portions 5033a to 5033c, and the like can be set as needed to provide the arm portion 5031 with desired degrees of freedom. For example, the arm portion 5031 can be suitably configured to have six degrees of freedom or higher. This enables to freely move the endoscope 5001 in a movable range of the arm portion 5031, so that the barrel 5003 of the endoscope 5001 can be inserted into the body cavity of the patient 5071 from desired directions.

The joint portions 5033a to 5033c include actuators, respectively, and the joint portions 5033a to 5033c are configured to be rotatable about predetermined rotational axes when driven by the actuators, respectively. The driving of the actuators is controlled by the arm control device 5045, whereby the rotation angles of the respective joint portions 5033a to 5033c are controlled to control the driving of the arm portion 5031. As a consequence, the control of the position and posture of the endoscope 5001 can be realized. Here, the arm control device 5045 can control the driving of the arm portion 5031 by various known control methods such as force control and position control.

For example, the operator 5067 may perform an operational input via the input device 5047 (including the foot switch 5057) as needed, whereby the driving of the arm portion 5031 may be suitably controlled in response to the operational input by the arm control device 5045 and the position and posture of the endoscope 5001 may be controlled. By the control, the endoscope 5001 on a distal end of the arm portion 5031 can be moved from a desired position to another desired position, and can then be fixedly supported at the position after the movement. It is to be noted that the arm portion 5031 may be operated by a so-called master-slave method. In this case, the arm portion 5031 can be remotely operated by the user via the input device 5047 arranged at a place remote from an operating room.

If force control is applied, on the other hand, the arm control device 5045 may receive an external force from the user and may drive the actuators of the respective joint portions 5033a to 5033c so that the arm portion 5031 smoothly moves according to the external force, in other words, so-called power assist control may be performed. As a consequence, when the user moves the arm portion 5031 while directly touching the arm portion 5031, the arm portion 5031 can be moved by a relatively light force. Therefore, the endoscope 5001 can be moved more intuitively by simpler operation, and the user's convenience can be improved.

Now, in surgery under endoscope, it has been a common practice to support the endoscope 5001 by a surgeon called “a scopist.” In contrast, the use of the support arm device 5027 enable to more steadily fix the position of the endoscope 5001 without relying upon manpower, so that an image of the operative field can be stably acquired and the surgery can be smoothly performed.

It is to be noted that the disposition of the arm control device 5045 on the cart 5037 is not absolutely needed. Further, the arm control device 5045 is not necessarily required to be a single device. For example, plural arm control devices 5045 may be disposed in the individual joint portions 5033a to 5033c, respectively, of the arm portion 5031 of the support arm device 5027, and drive control of the arm portion 5031 may be realized through mutual cooperation of the arm control devices 5045.

(Light Source Device)

The light source device 5043 supplies illumination light to the endoscope 5001 upon imaging the operative field. The light source device 5043 is configured, for example, from a white light source which is in turn configured by LEDs, laser light sources or a combination thereof. Now, in a case where a white light source is configured by a combination of RGB laser light sources, each color (each wavelength) can be controlled with high accuracy in output intensity and output timing, so that the white balance of an image to be captured can be adjusted at the light source device 5043. Further, in this case, images that correspond to RGB, respectively, can be captured by time sharing by illuminating laser light beams from respective RGB laser light sources to an observation target in a time division manner and controlling the driving of the imaging device in the camera head 5005 in synchronization with the timings of the illumination. According to this method, a color image can be acquired without disposing a color filter on the imaging device.

Further, the driving of the light source device 5043 may be controlled so that the intensity of light to be outputted is changed at predetermined time intervals. An image of high dynamic range, which is free of so-called blocked up shadows or blown out highlights, can be generated by controlling the driving of the imaging device in the camera head 5005 in synchronization with timings of the changes of the intensity of the light to acquire images in a time division manner and then combining the images.

Furthermore, the light source device 5043 may be configured to be able to supply light of a predetermined wavelength bandwidth corresponding to special light observation. In the special light observation, a predetermined tissue such as blood vessels in a mucosal surface layer is imaged with high contrast, in other word, so-called narrow band imaging is performed, for example, by using the wavelength dependency of absorption of light in a body tissue and illumination light of a bandwidth narrower than that of illumination light (specifically, white light) in normal observation. As an alternative, fluorescence observation may be performed in special light observation. According to the fluorescence observation, an image is acquired by fluorescence generated by illumination of excitation light. In fluorescence observation, it is possible to perform, for example, observation of fluorescence from a body tissue by illuminating excitation light to the body tissue (autofluorescence observation) or acquisition of a fluorescence image by locally injecting a reagent such as indocyanine green (ICG) or the like into a body tissue and illuminating excitation light, which corresponds to the wavelength of fluorescence from the reagent, to the body tissue. The light source device 5043 can be configured to be able to supply narrow band light and/or excitation light corresponding to such special light observation.

[Description of Configuration of Medical Observation System]

A description will next be made about the medical observation system 1000 that makes up a part of the endoscopic surgery system 5000. FIG. 2 is a functional block diagram depicting a functional configuration of the medical observation system 1000. The medical observation system 1000 includes an imaging apparatus 2000 making up a part of the camera head 5005, the CCU 5039, and the light source device 5043.

The imaging apparatus 2000 captures an image of the operative field in the body cavity of the patient 5071. The imaging apparatus 2000 includes a lens unit (unillustrated) and an imaging device 100. The lens unit is an optical system disposed in a connecting portion to the barrel 5003. Observed light introduced from the distal end of the barrel 5003 is guided to the camera head 5005, and then enters the lens unit. The lens unit is configured of a combination of plural lenses including a zoom lens and a focus lens. The lens unit has optical characteristics designed so that the observed light is condensed on a light-receiving surface of the imaging device 100.

The imaging device 100 is disposed in the casing, to which the barrel 5003 can be connected, at a later stage of the lens unit. The observed light which has passed through the lens unit condenses on the light-receiving surface of the imaging device 100, and image signals corresponding to the observed image are generated by photoelectric conversion. The image signals are supplied to the CCU 5039. As the imaging device 100 is, for example, an image sensor of the CMOS (Complementary Metal Oxide Semiconductor) type, and one having a Bayer array to enable capture of a color image is used.

Further, the imaging device 100 includes pixels, which receive normal light, and pixels, which receive special light. As operative field images acquired by imaging the operative field in the body cavity of the patient 5071, the imaging device 100 therefore captures a normal light image during illumination of normal light and a special light image during illumination of special light. The term “special light” as used herein means light of a predetermined wavelength bandwidth.

The imaging apparatus 2000 transmits image signals, which have been acquired from the imaging device 100, as RAW data to the CCU 5039. On the other hand, the imaging device 100 receives from the CCU 5039 control signals for controlling driving of the imaging apparatus 2000. The control signals include information regarding imaging conditions such as, for example, information to the effect that the frame rate of an image to be captured shall be specified, information to the effect that the exposure value upon imaging shall be specified, and/or information to the effect that the magnification and focal point of an image to be captured shall be specified, etc.

It is to be noted that the above-described imaging conditions, such as the frame rate, exposure value, magnification and focal point, are automatically set by a control section 5063 for the CCU 5039 based on acquired image signals. In other words, so-called AE (Auto Exposure) function, AF (Auto Focus) function and AWB (Auto White Balance) function are mounted on the endoscope 5001.

The CCU 5039 is an example of a signal processing apparatus. The CCU 5039 processes signals from the imaging device 100 that receive light guided from the barrel 5003, and transmits the processed signals to the display device 5041. The CCU 5039 includes a normal light development processing section 11, a special light development processing section 12, a three-dimensional information generating section 21, a three-dimensional information storage section 24, an interested region setting section 31, an estimated region calculating section 32, an image processing section 41, a display control section 51, an AE detection section 61, an AE control section 62, and a light source control section 63.

The normal light development processing section 11 performs development processing to convert RAW data, which have been acquired by imaging during illumination of normal light, to a visible image. The normal light development processing section 11 also applies a digital gain and a gamma curve to the RAW data to generate more conspicuous normal light image data.

The special light development processing section 12 performs development processing to convert RAW data, which have been acquired by imaging during illumination of special light, to a visible image. The special light development processing section 12 also applies a digital gain and a gamma curve to the RAW data to generate more conspicuous special light image data.

The three-dimensional information generating section 21 includes a map generation section 22 and a self-position estimation section 23. Based on RAW data outputted from the imaging apparatus 2000 or a normal light image captured during illumination of normal light such as normal light image data outputted from the normal light development processing section 11, the map generation section 22 generates three-dimensional information regarding the operative field in the body cavity. Described in more detail, the three-dimensional information generating section 21 generates three-dimensional information regarding the operative field from at least two sets of image data (operative field images) captured by imaging the operative field at different angles with the imaging apparatus 2000. For example, the three-dimensional information generating section 21 generates three-dimensional information by matching feature points in at least two sets of normal light image data. Here, the three-dimensional information includes, for example, three-dimensional map information with three-dimensional coordinates of the operative field represented therein, position information representing the position of the imaging apparatus 2000, and posture information representing the posture of the imaging apparatus 2000.

The map generation section 22 generates three-dimensional information by matching feature points in at least two sets of normal light image data. For example, the map generation section 22 extracts feature points, which correspond to the feature points contained in the image data, from three-dimensional map information stored in the three-dimensional information storage section 24. The map generation section 22 then generates three-dimensional map information by matching between the feature points contained in the image data and the feature points extracted from the three-dimensional map information. In addition, the map generation section 22 updates the three-dimensional map information as needed if image data have been captured. It is to be noted that a detailed generation method of three-dimensional map information will be described hereinafter.

The self-position estimation section 23 calculates the position and posture of the imaging apparatus 2000 based on RAW data or a normal light image captured during illumination of normal light, such as normal light image data, and the three-dimensional map information stored in the three-dimensional information storage section 24. For example, the self-position estimation section 23 calculates the position and posture of the imaging apparatus 2000 by differentiating which coordinates in the three-dimensional map information have feature points corresponding to the feature points contained in the image data. The self-position estimation section 23 then outputs position and posture information, which includes position information representing the position of the imaging apparatus 2000 and posture information representing the posture of the imaging apparatus 2000. It is to be noted that a detailed estimation method of self-position and posture will be described subsequently herein.

The three-dimensional information storage section 24 stores three-dimensional map information outputted from the map generation section 22.

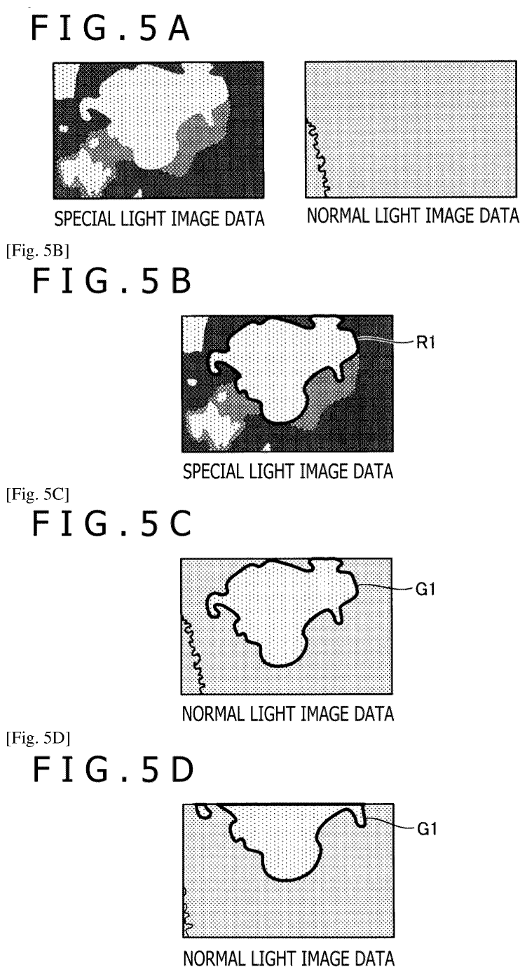

Based on the special light image data captured by the imaging apparatus 2000 during the illumination of the special light having the predetermined wavelength bandwidth, the interested region setting section 31 sets an interested region R1 (see FIG. 5B) in the special light image data. A feature region, which is a region characteristic by a feature value equal to or greater than a threshold in the special light image data, is set as the interested region R1. The interested region R1 means, for example, a region having a fluorescence intensity equal to or greater than the threshold in a case where a desired affected area is caused to emit fluorescence with a biomarker or the like.

Described in more detail, the interested region setting section 31 detects a feature region, which has a fluorescence intensity of the threshold or greater, from special light image data outputted from the special light development processing section 12 if an input instructing a timing, at which the interested region R1 is to be set, has been received via the input device 5047 or the like. The interested region setting section 31 then sets the feature region as the interested region R1. Further, the interested region setting section 31 specifies coordinates on a two-dimensional space, at which the interested region R1 has been detected in the special light image data. The interested region setting section 31 then outputs interested region coordinate information that represents the position, such as the coordinates, of the interested region R1 on the two-dimensional space in the special light image data.

The estimated region calculating section 32 estimates, from the three-dimensional information, an estimated region corresponding to the physical position of the interested region R1 in the normal light image data captured by the imaging apparatus 2000 during illumination of the normal light having the wavelength bandwidth different from the wavelength bandwidth of the special light. The estimated region calculating section 32 then outputs estimated region coordinate information representing the coordinates or the like of the estimated region on the two-dimensional space in the normal light image data.

Described in more detail, using the three-dimensional map information, the estimated region calculating section 32 calculates interested coordinates corresponding to the physical position of the interested region R1 at the three-dimensional coordinates, and based on the three-dimensional map information, position information and posture information, estimates as the estimated region a region corresponding to the interested coordinates in the normal light image data. In other words, the estimated region calculating section 32 calculates to which coordinates on the three-dimensional space in the three-dimensional map information the coordinates of the interested region R1 on the two-dimensional space as represented by the interested region coordinate information outputted from the interested region setting section 31 correspond. As a consequence, the estimated region calculating section 32 calculates the interested coordinates that represent the coordinates of the interested region R1 on the three-dimensional space. Further, if the three-dimensional information generating section 21 has outputted the position and posture information, the estimated region calculating section 32 calculates to which coordinates on the two-dimensional space of the normal light image data, which have been captured with the position and posture of the imaging apparatus 2000 as represented by the position and posture information, the interested coordinates of the interested region R1 on the three-dimensional space correspond. As a consequence, the estimated region calculating section 32 estimates, as an estimated region, the region corresponding to the physical position represented by the physical position of the interested region R1 in the normal light image data. The estimated region calculating section 32 then outputs estimated region coordinate information that represents the coordinates of the estimated region in the normal light image data.

Further, using machine learning, the estimated region calculating section 32 may automatically set the interested region R1 from the feature region contained in the special light image data, and may then set to which coordinates in the three-dimensional information such as the three-dimensional map information the interested region R1 corresponds.

The image processing section 41 applies predetermined image processing to the estimated region in the normal light image data. Based on the estimated region coordinate information representing the coordinates of the interested region R1, for example, the image processing section 41 performs image processing to superimpose annotation information G1 (see FIG. 5C), which represents features of the special light image data, on the estimated region in the normal light image data. In other words, the image processing section 41 applies image enhancement processing, which is different from that to be applied to an outside of the estimated region, to the estimated region. The term “image enhancement processing” means image processing that enhances an estimated region, for example, by the annotation information G1 or the like.

Described in more detail, the image processing section 41 generates display image data for the normal light image data. The display image data have been acquired by superimposing the annotation information G1, which was acquired by visualizing the interested region R1 in the special light image data, on the coordinates represented by the estimated region coordinate information. The image processing section 41 then outputs the display image data to the display control section 51. Here, the annotation information G1 is information in which the interested region R1 in the special light image data has been visualized. For example, the annotation information G1 is an image, which has the same shape as the interested region R1 and has been enhanced along the contour of the interested region R1. Further, the inside of the contour may be colored or may be transparent or translucent. Furthermore, the annotation information G1 may be generated, based on the special light image data outputted from the special light development processing section 12, by the image processing section 41, the interested region setting section 31, or a further function section.

The display control section 51 controls the display device 5041 to display a screen represented by the display image data.

Based on the estimated region coordinate information outputted from the estimated region calculating section 32, the AE detection section 61 extracts the respective interested regions R1 in the normal light image data and special light image data. From the respective interested regions R1 in the normal light image data and special light image data, the AE detection section 61 then extracts exposure information needed for an adjustment of the exposure. The AE detection section 61 thereafter outputs exposure information for the respective interested regions R1 in the normal light image data and special light image data.

The AE control section 62 controls AE functions. Descried in more detail, the AE control section 62, based on the exposure information outputted from the AE detection section 61, outputs control parameters, which include, for example, an analog gain and a shutter speed, to the imaging apparatus 2000.

Further, based on the exposure information outputted from the AE detection section 61, the AE control section 62 outputs control parameters, which include, for example, a digital gain and a gamma curve, to the special light development processing section 12. Furthermore, based on the exposure information outputted from the AE detection section 61, the AE control section 62 also outputs light quantity information, which represents the quantity of light to be illuminated by the light source device 5043, to the light source control section 63.

Based on the light quantity information outputted from the AE control section 62, the light source control section 63 controls the light source device 5043. The light source control section 63 then outputs light source control information to control the light source device 5043.

[Description of Generation Method of Three-Dimensional Map Information and Position and Posture Information]

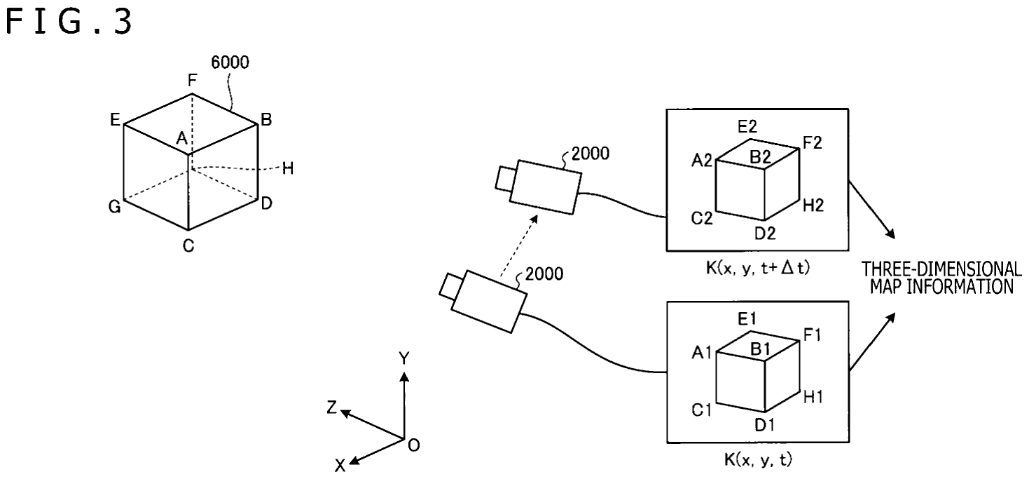

A description will next be made about a method that the three-dimensional information generating section 21 generates three-dimensional map information and position and posture information (information including the position information and posture information regarding the imaging apparatus 2000). FIG. 3 is a diagram illustrating a method that the three-dimensional information generating section 21 generates three-dimensional map information.

FIG. 3 illustrates how the imaging apparatus 2000 is observing a stationary object 6000 in a three-dimensional space XYZ with a point on the space serving as a reference position. It is now assumed that the imaging apparatus 2000 captured image data K(x,y,t) such as RAW data or normal light image data at time t and also image data K(x,y,t+Δt) such as RAW data or normal light image data at time t+Δt. It is to be noted that the time interval Δt is set, for example, at 33 msec or so. In addition, the reference position O may be set as desired, but is desirably set, for example, at a position that does not move with time. It is also to be noted that in the image data K(x,y,t), x represents a coordinate in a horizontal direction of the image and y represents a coordinate in a vertical direction of the image.

The map generation section 22 next detects feature points, which are characteristic pixels, out of the image data K(x,y,t) and the image data K(x,y,t+Δt). The term, “feature point” means, for example, a pixel having a pixel value different by a predetermined value or greater from that of the adjacent pixels. It is to be noted that the feature points are desirably points which stably exist even after an elapse of time, and that as the feature points, pixels defining edges in the images are frequently used, for example. To simplify the following description, it is now assumed that feature points A1, B1, C1, D1, E1, F1, and H1, which are apexes of the object 6000, have been detected out of the image data K(x,y,t).

The map generation section 22 next makes a search for points, which correspond to the feature points A1, B1, C1, D1, E1, F1, and H1, respectively, out of the image data K(x,y,t+Δt). Specifically, based on the pixel value of the feature point A1, pixel values in a vicinity of the feature point A1, and the like, a search is made for points having similar features out of the image data K(x,y,t+Δt). By this search processing, feature points A2, B2, C2, D2, E2, F2, and H2 corresponding to the feature points A1, B1, C1, D1, E1, F1, and H1 are detected, respectively, out of the image data K(x,y,t+Δt).

Based on the principle of three-dimensional surveying, the map generation section 22 subsequently calculates three-dimensional coordinates (XA,YA,ZA) of a point A on the space, for example, from the two-dimensional coordinates of the feature point A1 on the image data K(x,y,t+Δt) and the two-dimensional coordinates of the feature point A2 on the image data K(x,y,t+Δt). In this manner, the map generation section 22 generates, as a set of the calculated three-dimensional coordinates (XA,YA,ZA), three-dimensional map information regarding the space in which the object 6000 is placed. The map generation section 22 causes the three-dimensional information storage section 24 to store the generated three-dimensional map information. It is to be noted that the three-dimensional map information is an example of the three-dimensional information in the present disclosure.

In addition, the self-position estimation section 23 also estimates the position and posture of the imaging apparatus 2000 because the position and posture of the imaging apparatus 2000 have changed during the time interval Δt. Mathematically, taking, as unknowns, the three-dimensional coordinates of the respective feature points defining the object 6000 and the position and posture of the imaging apparatus 2000, simultaneous equations are established based on the two-dimensional coordinates of the feature points observed in the image data K(x,y,t) and image data K(x,y,t+Δt), respectively. The self-position estimation section 23 estimates the three-dimensional coordinates of the respective feature points defining the object 6000 and the position and posture of the imaging apparatus 2000 by solving the simultaneous equations.

By detecting the feature points, which correspond to the feature points detected from the image data K(x,y,t), from the image data K(x,y,t+Δt) (in other words, performing matching in feature points) as described above, the map generation section 22 generates three-dimensional map information regarding an environment under observation by the imaging apparatus 2000. Further, the self-position estimation section 23 can estimate the position and posture, in other words, self-position of the imaging apparatus 2000. Furthermore, the map generation section 22 can improve the three-dimensional map information by performing the above-described processing repeatedly, for example, to make feature points, which were invisible before, visible. Through the repeated processing, the map generation section 22 repeatedly calculates the three-dimensional positions of the same feature points, and therefore performs, for example, average processing so that calculation errors can be reduced. As a consequence, the three-dimensional map information stored in the three-dimensional information storage section 24 is updated continually. It is to be noted that the technique of generating the three-dimensional map information regarding the environment and specifying the self-position of the imaging apparatus 2000 by matching the feature points is generally called SLAM (Simultaneous Localization and Mapping) technique.

The fundamental principle of a SLAM technique with a monocular camera is described, for example, “Andrew J. Davison, “Real-Time Simultaneous Localization and Mapping with a Single Camera,” Proceedings of the 9th IEEE International Conference on Computer Vision, Volume 2, 2003, pp. 1403-1410.” Further, a SLAM technique that estimates the three-dimensional position of a subject by using a camera image of the subject is also called a Visual SLAM specifically.

[Description of Flow of Processing Performed by Medical Observation System According to First Embodiment]

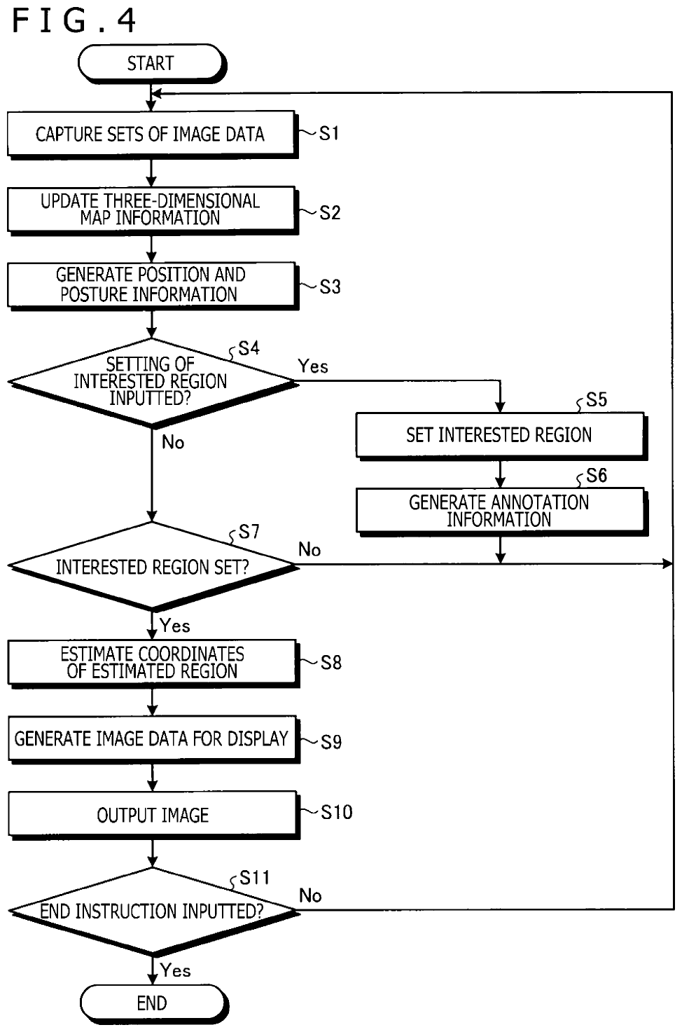

Referring next to FIGS. 4, 5A, 5B, 5C and 5D, a description will be made of a flow of processing that the medical observation system 1000 of the first embodiment performs. FIG. 4 is a flow chart illustrating an example of the flow of the processing that the medical observation system 1000 performs. FIG. 5A depicts images of examples of captured image data. FIG. 5B is an image depicting an example of an interested region R1 extracted from special light image data. FIG. 5C is an image depicting an example of display image data with annotation information G1 superimposed on normal light image data. FIG. 5D is an image depicting an example of display image data with the annotation information G1 superimposed on other normal light image data.

The imaging device 100 captures normal light image data and special light image data (step S1). For example, the imaging device 100 captures the normal light image data and special light image data depicted in FIG. 5A.

The three-dimensional information generating section 21 updates the three-dimensional map information based on the previous three-dimensional map information and normal light image data, and if necessary, based on captured normal light image data (step S2). If the region of the captured normal light image data is not included in the previously generated three-dimensional map information, for example, the three-dimensional information generating section 21 updates the three-dimensional map information. If the region of the captured normal light image data is included in the previously generated three-dimensional map information, on the other hand, the three-dimensional information generating section 21 does not update the three-dimensional map information.

Based on the captured normal light image data, the three-dimensional information generating section 21 generates position and posture information (step S3).

The interested region setting section 31 determines whether or not an instruction input to set the interested region R1 has been received (step S4).

If the instruction input to set the interested region R1 has been received (step S4: Yes), the interested region setting section 31 sets a feature region, which has been detected from the special light image data, as the interested region R1 (step S5). As depicted in FIG. 5B, for example, the interested region setting section 31 sets, as the interested region R1, a region caused to emit fluorescence at a fluorescence intensity of a threshold or higher with a biomarker or the like.

The image processing section 41 generates annotation information G1 based on the captured special light image data (step S6).

If the instruction input to set the interested region R1 has not been received in step S4 (step S4: No), the estimated region calculating section 32 determines whether or not the interested region R1 has been set (step S7). If the interested region R1 has not been set (step S7: No), the medical observation system 1000 causes the processing to return to step S1.

If the interested region R1 has been set on the other hand (step S7: Yes), the estimated region calculating section 32 estimates, from the three-dimensional information, the coordinates of an estimated region corresponding to the physical position of the interested region R1 in the captured normal light image data (step S8). In other words, the estimated region calculating section 32 calculates the coordinates of the estimated region.

The image processing section 41 generates display image data with image processing, such as superimposition of the annotation information G1, performed on the coordinates of the calculated estimated region in the normal light image data (step S9). As depicted in FIG. 5C, for example, the image processing section 41 generates display image data with the annotation information G1 superimposed on the normal light image data.

The display control section 51 outputs an image that the display image data represent (step S10). In other words, the display control section 51 causes the display device 5041 to display the image that the display image data represent.

The medical observation system 1000 determines whether or not an input to end the processing has been received (step S11). If an input to end the processing has not been received (step S11: No), the medical observation system 1000 causes the processing to return to step S1. In short, the medical observation system 1000 generates display image data with image processing, such as superimposition of the annotation information G1, performed on the calculated coordinates of the interested region R1 in the normal light image data captured again. As depicted in FIG. 5D, for example, it is therefore possible to generate display image data with the annotation information G1 superimposed on the coordinates of the interested region R1 even in normal light image data captured again in a state that the imaging apparatus 2000 has moved or has changed its posture.

If an input to end the processing has been received (step S11: Yes), the medical observation system 1000 then ends the processing.

As described above, the medical observation system 1000 according to the first embodiment sets an observation target, in other words, a feature region as the interested region R1. The medical observation system 1000 then performs predetermined image processing on an estimated region that has been estimated to correspond to a physical position representing a physical position of the interested region R1 in the normal light image data. For example, the medical observation system 1000 generates display image data with the annotation information G1, in which the interested region R1 has been visualized, superimposed thereon. As described above, even if the biomarker or the like has diffused or quenched, the medical observation system 1000 generates the display image data with the annotation information G1, that is, the visualized interested region R1 superimposed on the position of the estimated region which is estimated to be the position of the interested region R1. The medical observation system 1000 hence allows a user such as a surgeon to easily differentiate the observation target even after an elapse of time.

### Second Embodiment

In the above-described first embodiment, no limitation is imposed on the extraction of feature points in the generation of three-dimensional map information. In a second embodiment, an interested region R1 may be excluded from a region, from which feature points are to be extracted, if the interested region R1 has been set.

Here, the three-dimensional information generating section 21 is performing the generation and updating of three-dimensional map information based on feature points extracted from normal light image data. The accuracy of the three-dimensional map information therefore deteriorates if the positions of the feature points extracted from the normal light image data move.

The interested region R1 is a region in which a user such as a surgeon is interested, and is a target of treatment such as surgery, and therefore has high possibility of deformation. Accordingly, the extraction of feature points from the interested region R1 leads to a high possibility of deteriorating the accuracy of the three-dimensional map information. If the interested region setting section 31 has set the interested region R1, the three-dimensional information generating section 21 hence extracts feature points from an outside of the interested region R1 represented by the interested region coordinate information. The three-dimensional information generating section 21 then updates the three-dimensional map information based on the feature points extracted from the outside of the interested region R1.

### Third Embodiment

In the above-described first embodiment, no limitation is imposed on the extraction of feature points in the generation of three-dimensional map information. In a third embodiment, a target such as a predetermined tool or the like is excluded from a target from which feature points are to be extracted.

For example, surgical instruments such as a scalpel and forceps 5023 are frequently inserted into, taken out of, and moved in an operative field. If the generation or updating of the three-dimensional map information is performed based on feature points extracted from a specific tool such as the scalpel or the forceps 5023, there is an increasing possibility that the accuracy of the three-dimensional map information may deteriorate. The three-dimensional information generating section 21 therefore excludes a predetermined tool from an extraction target for feature points.

Described in more detail, the three-dimensional information generating section 21 detects a predetermined tool such as the scalpel or the forceps 5023 from the normal light image data by pattern matching or the like. The three-dimensional information generating section 21 detects feature points from a region other than the region in which the predetermined tool has been detected. The three-dimensional information generating section 21 then updates the three-dimensional map information based on the extracted feature points.

### Fourth Embodiment

In the above-described first embodiment, display image data are outputted with annotation information G1, which has been acquired by visualizing an interested region R1 in special light image data, superimposed on the coordinates of an estimated region that is estimated to correspond to the physical position of an interested region R1 in normal light image data. In a fourth embodiment, display image data are outputted with not only information, which has been acquired by visualizing an interested region R1 in special light image data, but also annotation information G1, to which information regarding the interested region R1 has been added, being superimposed.

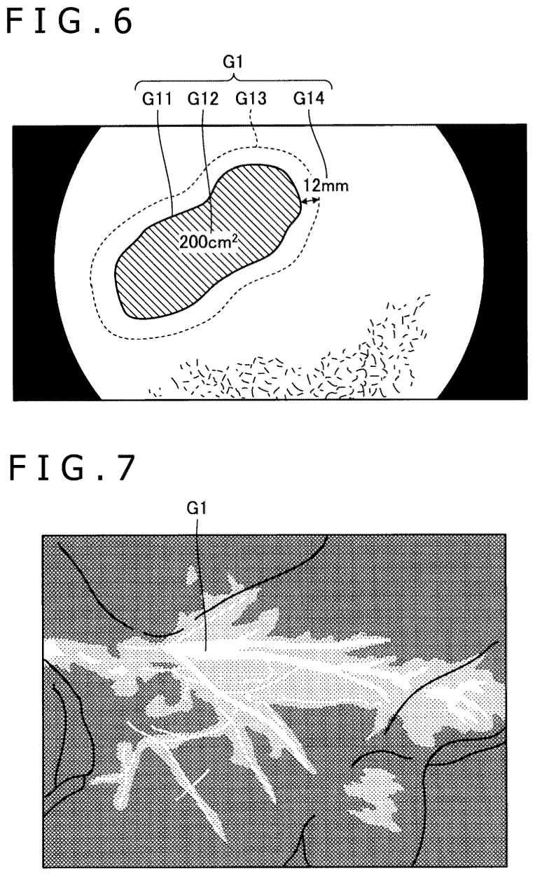

FIG. 6 is an image depicting an example of display image data with the annotation information G1, to which the information regarding the interested region R1 has been added, superimposed thereon. FIG. 6 depicts display image data with the annotation information G1 superimposed on an estimated region that is estimated to correspond to the physical position of an interested region R1 detected from an organ included in an operative field. As also depicted in FIG. 6, in the display image data, the annotation information G1 with information added thereto regarding the interested region R1 has been added to the estimated region in the normal light image data.

Described in more detail, the annotation information G1 depicted in FIG. 6 includes interested region information G11, area size information G12, boundary line information G13, and distance-to-boundary information G14. The interested region information G11 is information that represents the position and shape of the interested region R1. The area size information G12 is information that represents the area size of the interested region R1. The boundary line information G13 is information that represents whether or not a boundary line is inside a region widened by a preset distance from a contour of the interested region R1. The distance-to-boundary information G14 is information representing the preset distance in the boundary line information G13. By adding the information as described above, a user such as a surgeon can easily grasp a target of treatment or the like if an affected area extending over a specific distance from the interested region R1 is taken as the target of treatment or the like. It is to be noted that the preset distance is a value which can be changed as desired. Further, it can be changed as desired whether or not the area size value and distance value are displayed.

### Fifth Embodiment

In a fifth embodiment, display image data are outputted with annotation information G1 superimposed according to feature values of an interested region R1. The medical observation system 1000 outputs, for example, display image data with annotation information G1 superimposed according to fluorescence intensities of respective regions included in a fluorescent region.

Now, FIG. 7 is an image depicting an example of display image data with annotation information G1 superimposed thereon corresponding to feature values of every region contained in an interested region R1. The display image data depicted in FIG. 7 are for use in observing the state of blood vessels that are emitting fluorescence owing to a biomarker injected therein.

In more detail, the imaging device 100 captures an image of blood vessels that are emitting fluorescence owing to a biomarker injected therein by illuminating special light. The special light development processing section 12 generates special light image data of the blood vessels that are emitting fluorescence owing to the biomarker. The interested region setting section 31 extracts a feature region from the generated special light image data. The interested region setting section 31 then sets the feature region, in other words, the fluorescent region of the blood vessels as the interested region R1. Further, the image processing section 41 extracts fluorescence intensity at every pixel in the set interested region R1. Based on the fluorescence intensities in the interested region R1, the image processing section 41 generates display image data with annotation information G1, which corresponds to the fluorescent intensities of the respective pixels, superimposed on an estimated region that is estimated to correspond to the physical position of the interested region R1. Here, the expression “the annotation information G1, which corresponds to the fluorescence intensities” may mean annotation information G1 in which the hue, saturation and brightness differ at each pixel depending on the fluorescence intensity of the corresponding pixel, or annotation information G1 in which the luminance differs at each pixel depending on the fluorescence intensity of the corresponding pixel.

### Sixth Embodiment

In a sixth embodiment, display image data are outputted with annotation information G1, which is based on feature values of an interested region R1, being superimposed. Using a laser speckle method, a biomarker or the like, for example, the medical observation system 1000 can differentiate a state of blood, specifically an area where a blood flow exists. In the case of an image representing special light image data, the medical observation system 1000 sets, as an interested region R1, a location where a blood flow is abundant. Based on the feature quantities of the interested region R1, the medical observation system 1000 then superimposes annotation information G1, which represents the state of blood, specially a blood flow rate, on normal light image data.

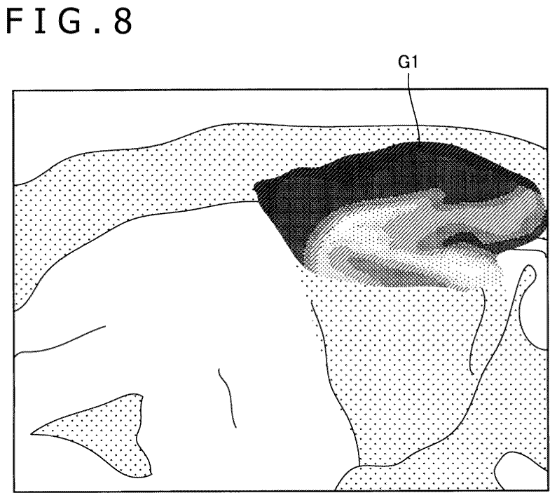

FIG. 8 is an image depicting an example of the display image data with the annotation information G1, which represents the blood flow, superimposed thereon. In the display image data depicted in FIG. 8, the annotation information G1, which represents the state of blood, specifically the blood flow rate in a pool of blood, has been superimposed.

In more detail, the special light development processing section 12 generates special light image data that represent the state of blood, specifically the blood flow. Based on the state of blood at a surgical area, the interested region setting section 31 sets the interested region R1. For example, the interested region setting section 31 sets, as the interested region R1, a region where the blood flow is estimated to be more abundant than a threshold. On the other hand, the estimated region calculating section 32 calculates the coordinates of an estimated region which has been estimated to correspond to the physical position of an interested region R1 in normal light image data.

Based on feature values of the interested region R1 in the special light image data, the image processing section 41 generates annotation information G1 that represents the state of blood. Based on the feature values of the interested region R1, the image processing section 41 generates, for example, annotation information G1 that expresses, in a pseudo color, the blood flow in the interested region R1. Specifically, the image processing section 41 generates the annotation information G1 with the blood flow expressed in terms of hue, saturation and brightness. As an alternative, the image processing section 41 generates the annotation information G1 by cutting out the interested region R1 in the case where the special light image data are in the form of an image with the blood flow expressed in a pseudo color.

The image processing section 41 superimposes the annotation information G1, in which the blood flow is expressed in the pseudo color, on the coordinates of the estimated region that is estimated to correspond to the physical position of the interested region R1 in the normal light image data. In the manner as described above, the image processing section 41 generates display image data with the annotation information G1, which represents the state of blood, specifically the blood flow rate, being superimposed thereon. A user such as a surgeon can easily grasp a location, where the blood flow is abundant, by watching the display image data with the annotation information G1, which represents the blood flow, superimposed thereon.

### Seventh Embodiment

In the above-described first embodiment, display image data are generated by image processing such as the superimposition of annotation information G1 on normal light image data. In a seventh embodiment, display image data are generated by image processing such as the superimposition of annotation information G1 on three-dimensional map information.

In more detail, the image processing section 41 generates display image data by image processing such as the superimposition of the annotation information G1 on the three-dimensional map information rather than normal light image data. For example, the image processing section 41 generates display image data by image processing such as the superimposition of the annotation information G1 on three-dimensional map information in which the distance from the imaging apparatus 2000 to a subject is expressed in a pseudo color. As a consequence, a user such as a surgeon can grasp the distance to the interested region R1 more exactly.

### Eighth Embodiment

In the above-described first embodiment, display image data are generated by image processing such as the superimposition of annotation information G1, which has been generated based on feature values upon setting as an interested region R1, on normal light image data. In an eighth embodiment, display image data are generated by image processing such as the superimposition of annotation information G1, which has been updated as needed, on normal light image data.

If the input device 5047 has received an operation, if a preset period of time has elapsed, or if a predetermined condition has been detected from image data such as special light image data or normal light image data, for example, the image processing section 41 updates annotation information G1 based on the feature values of an interested region R1 at that time. The image processing section 41 then generates display image data by image processing such as the superimposition of the updated annotation information G1 on the normal light image data. As a consequence, a user such as a surgeon can grasp how the interested region R1 changes with time. The user such as the surgeon can grasp, for example, how a biomarker diffuses or the like with time.

It is to be noted that the interested region setting section 31 may update the setting of the interested region R1 upon updating the annotation information G1. In this case, upon updating the annotation information G1, the interested region setting section 31 sets a newly extracted feature region as the interested region R1. Further, the estimated region calculating section 32 estimates an estimated region that corresponds to the physical position of the newly set interested region R1. The image processing section 41 then performs image processing, such as the superimposition of the annotation information G1, on the estimated region which has been newly estimated.

### Ninth Embodiment

In the above-described first embodiment, a feature region in special light image data is set as an interested region R1. In a ninth embodiment, an instruction to set as the interested region R1 is received. Described specifically, if one or more feature regions are detected from special light image data, the interested region setting section 31 provisionally sets the detected one or more feature regions as an interested region R1. Further, the interested region setting section 31 sets, as a formal interested region R1, the selected interested region R1 of the interested regions R1 set provisionally. The image processing section 41 then performs image processing such as the superimposition of annotation information G1 on an estimated region that is estimated to correspond to the physical position of the formal interested region R1.

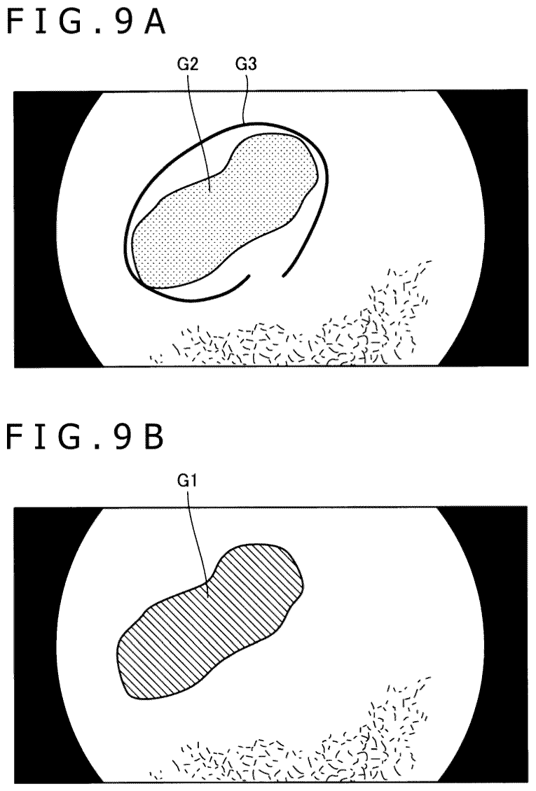

FIG. 9A is an image depicting an example of a method for specifying an interested region R1. FIG. 9B is an image depicting an example of setting of an interested region R1. FIG. 9A depicts display image data with provisional annotation information G2, which has been acquired by visualizing the interested region R1 provisionally set as the interested region R1, superimposed on normal light image data. FIG. 9A also depicts a specifying line G3 that surrounds the provisional annotation information G2. As depicted in FIG. 9B, the feature region, which is located inside the specifying line G3 and has been provisionally set as the interested region R1, is set as the formal interested region R1.

It is to be noted that an operation to specify the interested region R1 may be received on an image represented by special light image data. In addition, the method of specifying the interested region R1 is not limited to the operation to surround the provisional annotation information G2. For example, the interested region R1 may be specified by an operation to click the provisional annotation information G2, the provisional annotation information G2 may be specified by numerical values representing coordinates, or the provisional annotation information G2 may be specified by a name representing an affected area.

In more detail, if one or more feature regions are extracted, the interested region setting section 31 sets the extracted one or more feature regions as an interested region R1 provisionally. The estimated region calculating section 32 then outputs estimated region coordinate information representing the coordinates of an estimated region that is estimated to correspond to the physical position or the physical positions of the one or more interested regions R1 set provisionally. The image processing section 41 generates display image data for display purpose with the provisional annotation information G2, which has been obtained by visualizing the provisionally set interested region R1, superimposed on the coordinates represented by the estimated region coordinate information in normal light image data.

If the input device 5047 or the like has received an operation or the like to select the provisional annotation information G2, the interested region setting section 31 then cancels the setting of the provisional interested region R1 with respect to any unselected feature region. The estimated region calculating section 32 then outputs estimated region coordinate information representing the coordinates of the estimated region that is estimated to correspond to the physical position of the selected interested region R1. The image processing section 41 generates display image data for display purpose, with image processing, such as the superimposition of annotation information G1 on the coordinate represented by the estimated region coordinate information, performed on the normal light image data. The annotation information G1 has been acquired by visualizing the feature values in the special light image data. As a consequence, the image processing section 41 deletes the provisional annotation information G2 regarding the unselected feature region, and causes to display the annotation information G1 regarding the selected interested region R1. It is to be noted that the image processing section 41 may display the unselected feature region and the selected interested region R1 differentiably, without being limited to the deletion of the provisional annotation information G2 regarding the unselected feature regions.

### Tenth Embodiment

In the first embodiment, the medical observation system 1000 was described as one including the imaging apparatus 2000 with the imaging device 100 that receives both normal light and special light. In a tenth embodiment, a medical observation system 1000a includes an imaging apparatus 2000a having an imaging device 100 that receives normal light and a special light imaging device 200 that receives special light.

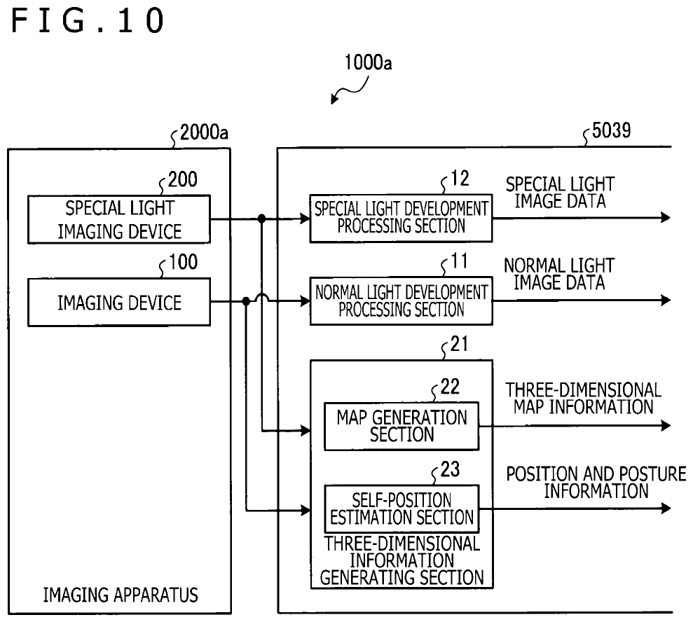

FIG. 10 is a diagram depicting an example of a configuration of a part of the medical observation system 1000a according to the tenth embodiment. The imaging apparatus 2000 includes both the imaging device 100 for normal light and the special light imaging device 200 for special light. In this case, the light source device 5043 may always illuminate both normal light and special light, or may alternately illuminate normal light and special light by changing them every time a predetermined period of time elapses.

### Eleventh Embodiment

In the first embodiment, the medical observation system 1000 was described to generate three-dimensional information based on image data captured by the imaging device 100. In an eleventh embodiment, a medical observation system 1000b generates three-dimensional information by using depth information acquired from an imaging and phase-difference sensor 120.

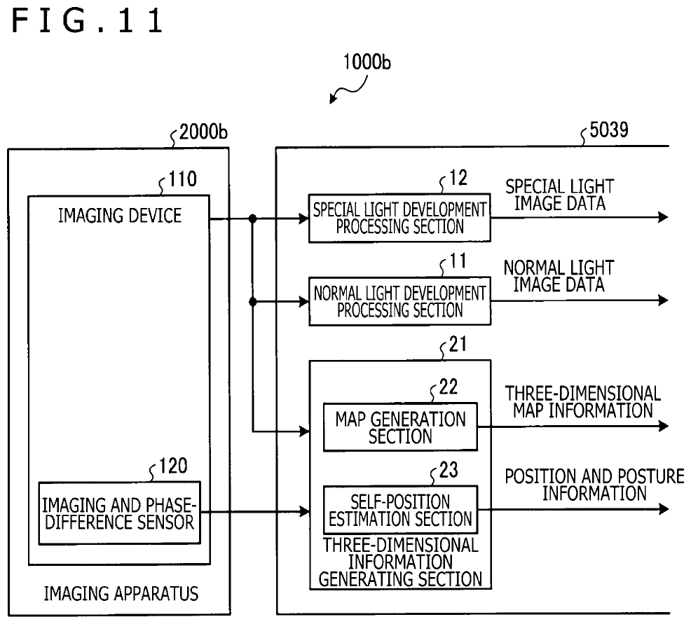

FIG. 11 is a diagram depicting an example of a configuration of a part of the medical observation system 1000b according to the eleventh embodiment. It is to be noted that FIG. 11 depicts FIG. 2 with a part thereof omitted, and the omitted part has the same configuration as in FIG. 2 unless otherwise specifically indicated.

An imaging apparatus 2000b includes an imaging device 110 having the imaging and phase-difference sensor 120. The imaging and phase-difference sensor 120 has a configuration that pixels, which measure the distance to a subject, are discretely arranged in the imaging device 110. The three-dimensional information generating section 21 acquires distance information regarding an operative field from the imaging and phase-difference sensor 120, and generates three-dimensional information by matching the feature points regarding the distance information. Described in more detail, the three-dimensional information generating section 21 captures, from imaging and phase-difference information outputted from the imaging and phase-difference sensor 120, depth information (distance information) from the imaging apparatus 2000b to the subject. Using the depth information (distance information), the three-dimensional information generating section 21 generates three-dimensional information such as three-dimensional map information through effective use of a SLAM technique. It is to be noted that the imaging and phase-difference sensor 120 can acquire depth information from an captured single set of image data. Further, the medical observation system 1000b can acquire depth information from a single captured image, and therefore can measure the three-dimensional position of a subject with high accuracy even if the subject is moving.

### Twelfth Embodiment

In the eleventh embodiment, the medical observation system 1000b was described as one including the imaging apparatus 2000b with the imaging device 110 that receives both normal light and special light. In a twelfth embodiment, a medical observation system 1000c includes both the imaging device 110 for normal light and the special light imaging device 200 for special light.

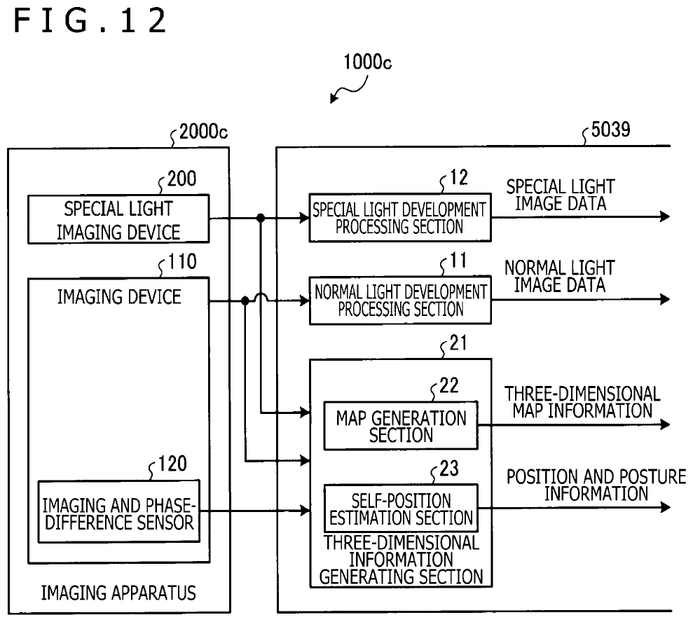

FIG. 12 is a diagram depicting an example of a configuration of a part of the medical observation system 1000c according to the twelfth embodiment. It is to be noted that FIG. 12 depicts FIG. 2 with a part thereof omitted, and the omitted part has the same configuration as in FIG. 2 unless otherwise specifically indicated. Further, the medical observation system 1000c according to the twelfth embodiment is different from the medical observation system 1000b according to the eleventh embodiment in that the medical observation system 1000c includes both the imaging device 110 for normal light and the special light imaging device 200 for special light. An imaging apparatus 2000c therefore includes the imaging device 110 for normal light, which has the imaging and phase-difference sensor 120, and the special light imaging device 200 for special light.

### Thirteenth Embodiment

In a thirteenth embodiment, a medical observation system 1000d includes an imaging apparatus 2000d having two imaging devices 100 and 101. In other words, the medical observation system 1000d includes a stereo camera.

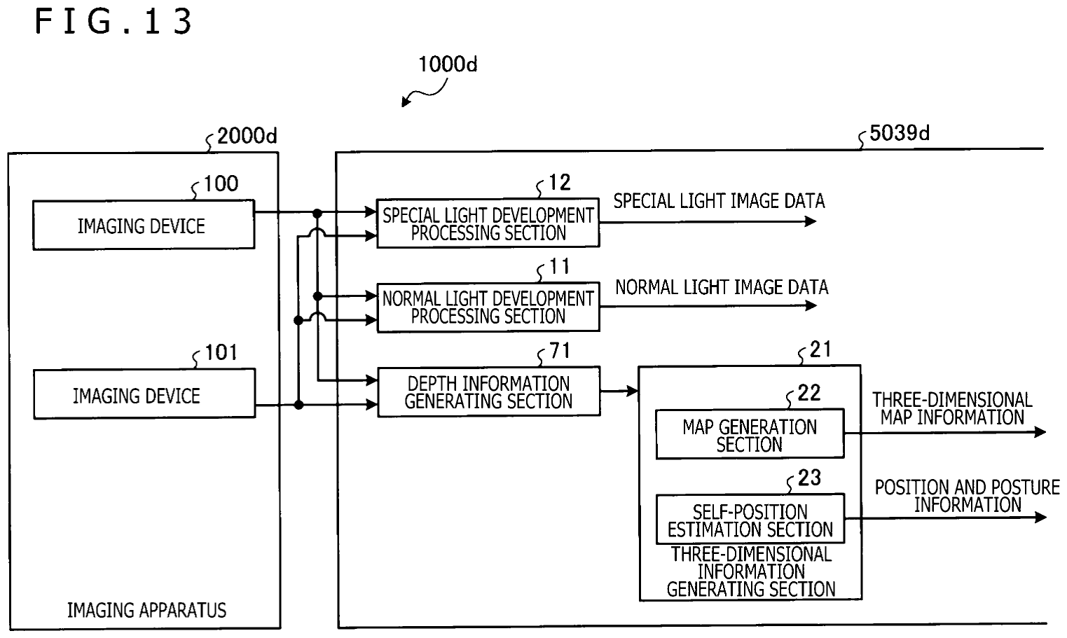

FIG. 13 is a diagram depicting an example of a configuration of a part of the medical observation system 1000d according to the thirteenth embodiment. It is to be noted that FIG. 13 depicts FIG. 2 with a part thereof omitted, and the omitted part has the same configuration as in FIG. 2 unless otherwise specifically indicated.

The two imaging devices 100 and 101 capture an image of different subjects, which are arranged in a state that they maintain a predetermined relative relationship, so that they overlap each other in parts. For example, the imaging devices 100 and 101 acquire image signals for the right eye and the left eye, respectively, so that stereovision is possible.

In the medical observation system 1000d, CCU 5039d also includes the depth information generating section 71 in addition to the configuration described with reference to FIG. 2. The depth information generating section 71 generates depth information by matching the feature points of two sets of image data captured by the respective two imaging devices 100 and 101.

Based on the depth information generated by the depth information generating section 71 and the image data captured by the respective imaging devices 100 and 101, the map generation section 22 generates three-dimensional information such as three-dimensional map information by using a SLAM technique. Further, the two imaging devices 100 and 101 can perform imaging at the same time, so that the depth information can be obtained from two images obtained by performing imaging once. The medical observation system 1000d can therefore measure the three-dimensional position of a subject even if the subject is moving.

### Fourteenth Embodiment

In the thirteenth embodiment, the medical observation system 1000d was described as one including an imaging apparatus 2000d with the imaging devices 100 and 101 that receive both normal light and special light. In a fourteenth embodiment, a medical observation system 1000e includes both the imaging devices 100 and 101 for normal light and the special light imaging devices 200 and 201 for special light.

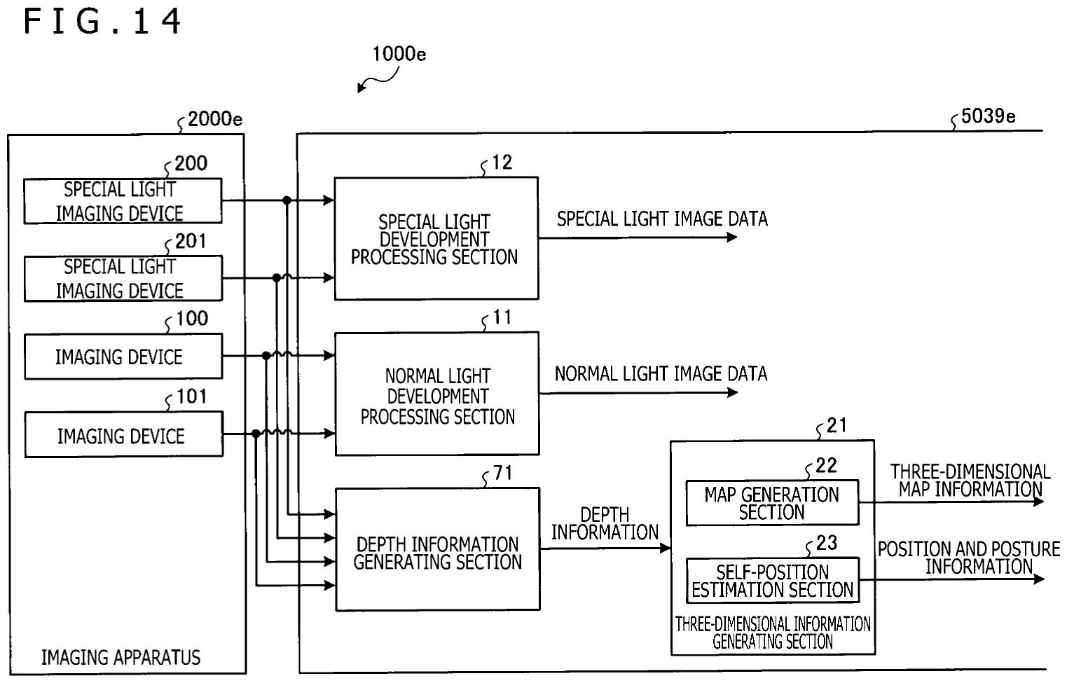

FIG. 14 is a diagram depicting an example of a configuration of a part of the medical observation system 1000e according to the fourteenth embodiment. It is to be noted that FIG. 14 depicts FIG. 2 with a part thereof omitted, and the omitted part has the same configuration as in FIG. 2 unless otherwise specifically indicated. Further, the medical observation system 1000e according to the fourteenth embodiment is different from the medical observation system 1000d according to the thirteenth embodiment in that the medical observation system 1000e includes both the imaging devices 100 and 101 for normal light and the special light imaging devices 200 and 201 for special light. An imaging apparatus 2000e therefore includes the two imaging devices 100 and 101 for normal light and the two special light imaging devices 200 and 201. In addition, CCU 5039e includes the depth information generating section 71.

### Fifteenth Embodiment

In a fifteenth embodiment, a medical observation system 1000f specifies an interested region R1 by tracking.

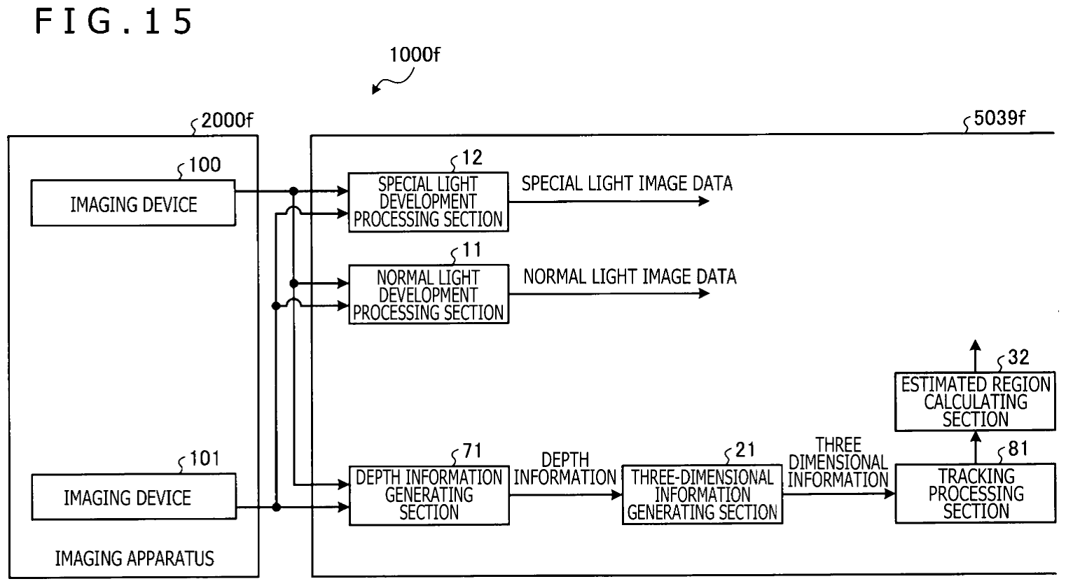

FIG. 15 is a diagram depicting an example of a configuration of a part of the medical observation system 1000f according to the fifteenth embodiment. It is to be noted that FIG. 15 depicts FIG. 2 with a part thereof omitted, and the omitted part has the same configuration as in FIG. 2 unless otherwise specifically indicated.

An imaging apparatus 2000f includes the two imaging devices 100 and 101, in other words, a stereo camera. CCU 5039f further includes the depth information generating section 71 and a tracking processing section 81. The depth information generating section 71 generates depth information by matching the feature points in two sets of image data captured by the respective two imaging devices 100 and 101.

Based on the depth information generated by the depth information generating section 71, the three-dimensional information generating section 21 generates three-dimensional map information. Based on three-dimensional information regarding an immediately preceding frame and three-dimensional information regarding a current frame, the tracking processing section 81 calculates differences in the position and posture of the imaging apparatus 2000f by using an IPC (Iterative Closest Point) method, which is a method that matches two clouds of points, or a like method. Based on the difference values in the position and posture of the imaging apparatus 2000f as calculated by the tracking processing section 81, the estimated region calculating section 32 calculates the coordinates of an estimated region on a two-dimensional screen. The image processing section 41 then generates display image data for display purpose with annotation information G1, which has been acquired by visualizing the feature values of special light image data, superimposed on the coordinates in normal light image data as calculated by the tracking processing section 81.

### Sixteenth Embodiment

In the fifteenth embodiment, the medical observation system 1000f was described as one including the imaging apparatus 2000f with the imaging devices 100 and 101 that receive both normal light and special light. In a sixteenth embodiment, a medical observation system 1000g includes both the imaging devices 100 and 101 for normal light and the special light imaging devices 200 and 201 for special light.

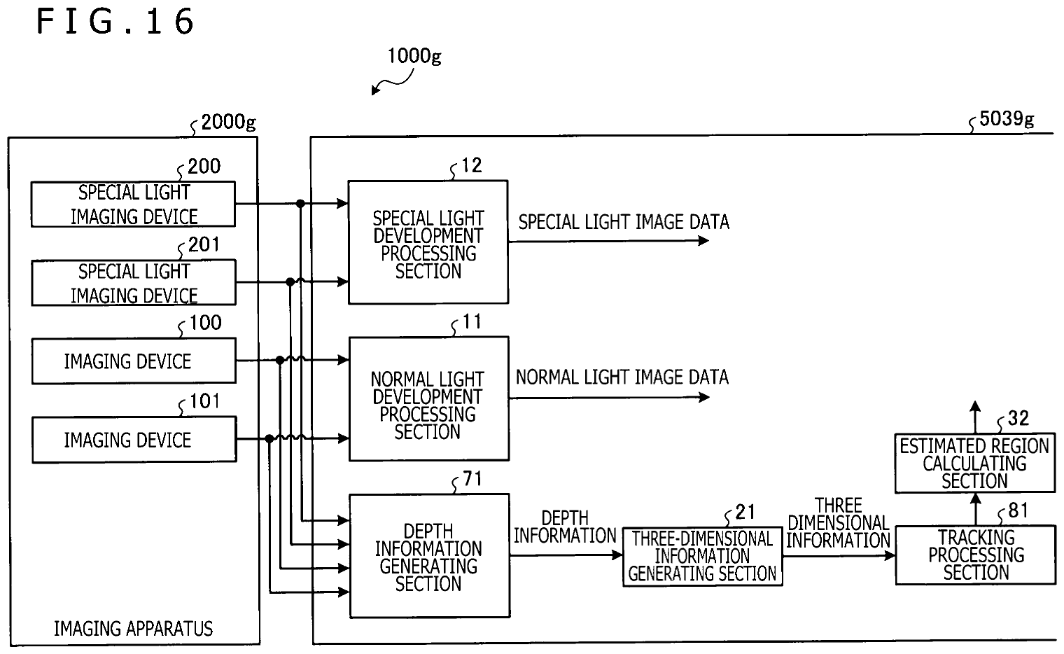

FIG. 16 is a diagram depicting an example of a configuration of a part of the medical observation system 1000g according to the sixteenth embodiment. It is to be noted that FIG. 16 depicts FIG. 2 with a part thereof omitted, and the omitted part has the same configuration as in FIG. 2 unless otherwise specifically indicated. Further, the medical observation system 1000g according to the sixteenth embodiment is different from the medical observation system 1000f according to the fifteenth embodiment in that the medical observation system 1000g includes both the imaging devices 100 and 101 for normal light and the special light imaging devices 200 and 201 for special light. An imaging apparatus 2000g therefore includes the two imaging devices 100 and 101 for normal light and the two special light imaging devices 200 and 201 for special light. In addition, CCU 5039g includes the depth information generating section 71 and the tracking processing section 81. Further, the medical observation system 1000g specifies an interested region R1 by tracking.

### Seventeenth Embodiment

In a seventeenth embodiment, a medical observation system 1000h generates three-dimensional information such as three-dimensional map information by a depth sensor 300.

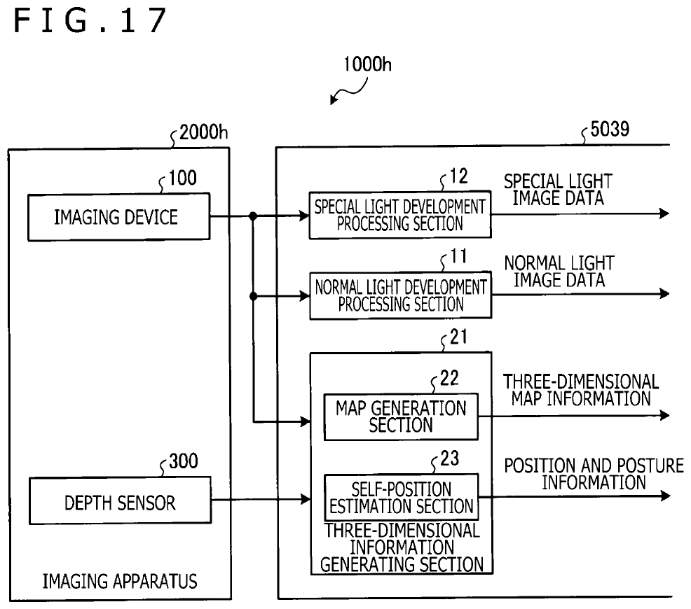

FIG. 17 is a diagram depicting an example of a configuration of a part of the medical observation system 1000h according to the seventeenth embodiment. It is to be noted that FIG. 17 depicts FIG. 2 with a part thereof omitted, and the omitted part has the same configuration as in FIG. 2 unless otherwise specifically indicated. An imaging apparatus 2000h includes the imaging device 100 and the depth sensor 300.

The depth sensor 300 is a sensor that measures a distance to a subject. The depth sensor 300 is, for example, a ToF (Time of Flight) sensor that measures the distance to the subject by receiving reflected light such as infrared light or the like illuminated toward the subject and measuring the time of flight of the light. It is to be noted that the depth sensor 300 may be realized by a structured light projection method. The structured light projection method measures the distance to the subject by capturing an image of projected light having a plurality of different geometric patterns and illuminated on the subject.

The map generation section 22 generates three-dimensional information by acquiring, from the depth sensor 300, distance information regarding an operative field and matching feature points in the distance information. More specifically, the map generation section 22 generates three-dimensional map information based on image data captured by the imaging device 100 and depth information (distance information) outputted by the depth sensor 300. For example, the map generation section 22 calculates to which pixels in the image data, which have been captured by the imaging device 100, points ranged by the depth sensor 300 correspond. The map generation section 22 then generates the three-dimensional map information regarding the operative field. Using the depth information (distance information) outputted from the depth sensor 300, the map generation section 22 generates the three-dimensional map information by a SLAM technique as described above.

### Eighteenth Embodiment

In the seventeenth embodiment, the medical observation system 1000h was described as one including the imaging apparatus 2000h with the imaging device 100 that receives both normal light and special light. In an eighteenth embodiment, a medical observation system 1000i includes both the imaging device 100 for normal light and the special light imaging device 200 for special light.

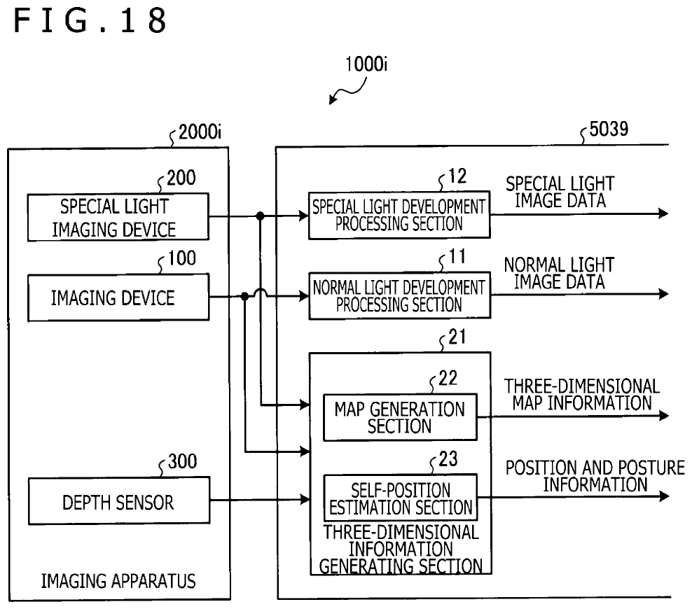

FIG. 18 is a diagram depicting an example of a configuration of a part of the medical observation system 1000i according to the eighteenth embodiment. It is to be noted that FIG. 18 depicts FIG. 2 with a part thereof omitted, and the omitted part has the same configuration as in FIG. 2 unless otherwise specifically indicated. Further, the medical observation system 1000i according to the eighteenth embodiment is different from the medical observation system 1000h according to the seventeenth embodiment in that the medical observation system 1000i includes both the imaging device 100 for normal light and the special light imaging device 200 for special light. An imaging apparatus 2000i therefore includes the imaging device 100 for normal light, the special light imaging device 200 for special light, and the depth sensor 300.

### Nineteenth Embodiment

In a nineteenth embodiment, a medical observation system 1000j specifies the coordinates of an interested region R1 through tracking by using three-dimensional information outputted by the depth sensor 300.

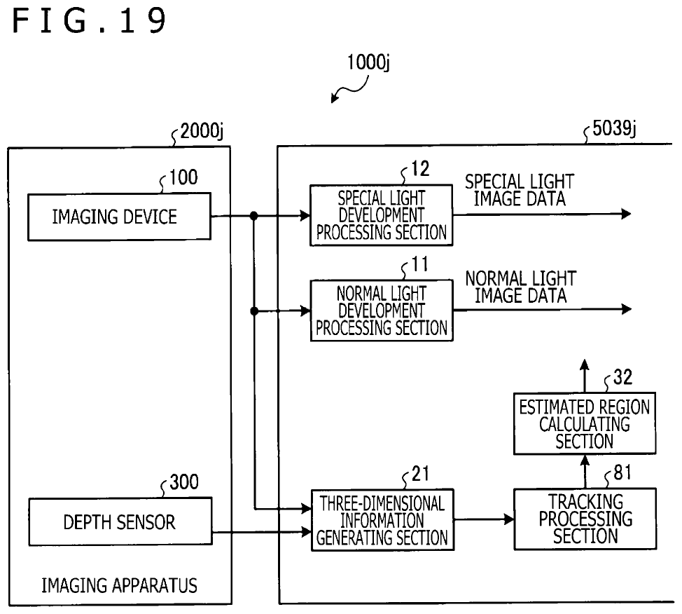

FIG. 19 is a diagram depicting an example of a configuration of a part of the medical observation system 1000j according to the nineteenth embodiment. It is to be noted that FIG. 19 depicts FIG. 2 with a part thereof omitted, and the omitted part has the same configuration as in FIG. 2 unless otherwise specifically indicated. An imaging apparatus 2000j includes the imaging device 100 and depth sensor 300. On the other hand, CCU 5039j further includes the tracking processing section 81.

The three-dimensional information generating section 21 generates three-dimensional information by acquiring, from the depth sensor 300, distance information regarding an operative field and performing matching with feature points in the distance information. More specifically, the three-dimensional information generating section 21 determines a moved state of a subject by matching two pieces of distance information (for example, distance images in which pixel values corresponding to the distances to the subject are stored) measured from different positions by the depth sensor 300. It is to be noted that the matching may preferably be performed between feature points themselves. Based on the moved state of the subject, the tracking processing section 81 calculates differences in the position and posture of the imaging apparatus 2000j. Based on the difference values in the position and posture of the imaging apparatus 2000j as calculated by the tracking processing section 81, the estimated region calculating section 32 calculates the coordinates of an estimated region on a two-dimensional screen. The image processing section 41 then generates display image data for display purpose with annotation information G1, which has been acquired by visualizing the feature values of special light image data on the coordinates calculated by the tracking processing section 81, superimposed on normal light image data.

### Twentieth Embodiment

In the nineteenth embodiment, the medical observation system 1000j was described as one including the imaging apparatus 2000j with the imaging device 100 that receives both normal light and special light. In a twentieth embodiment, a medical observation system 1000k includes both the imaging device 100 for normal light and the special light imaging device 200 for special light.

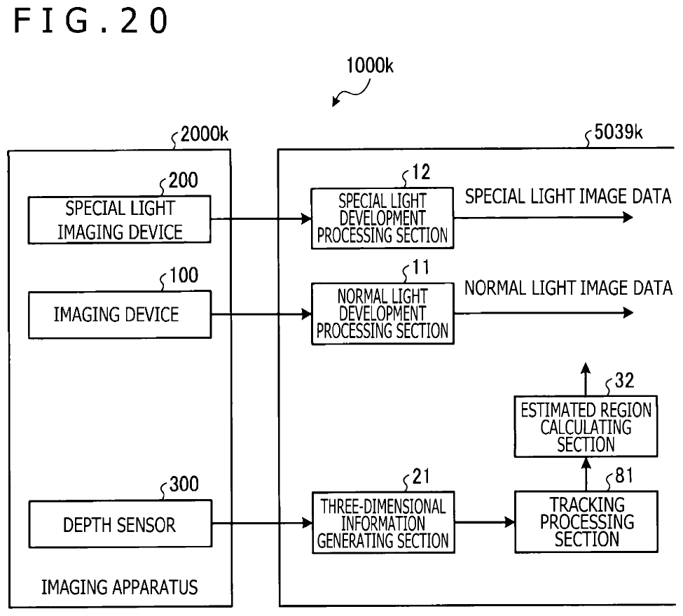

FIG. 20 is a diagram depicting an example of a configuration of a part of the medical observation system 1000k according to the twentieth embodiment. It is to be noted that FIG. 20 depicts FIG. 2 with a part thereof omitted, and the omitted part has the same configuration as in FIG. 2 unless otherwise specifically indicated. Further, the medical observation system 1000k according to the twentieth embodiment is different from the medical observation system 1000i according to the nineteenth embodiment in that the medical observation system 1000k includes both the imaging device 100 for normal light and the special light imaging device 200 for special light. An imaging apparatus 2000k therefore includes the imaging device 100 for normal light, the special light imaging device 200 for special light, and the depth sensor 300. On the other hand, CCU 5039k further includes the tracking processing section 81. Further, the medical observation system 1000k specifies the coordinates of an interested region R1 through matching.

### Twenty-First Embodiment

Techniques according to the present disclosure can be applied to a variety of products. For example, one or more of the techniques according to the present disclosure may be applied to a microscopic surgery system used in surgery to be performed while observing an infinitesimal site of a patient under magnification, in other words, in so-called microsurgery.

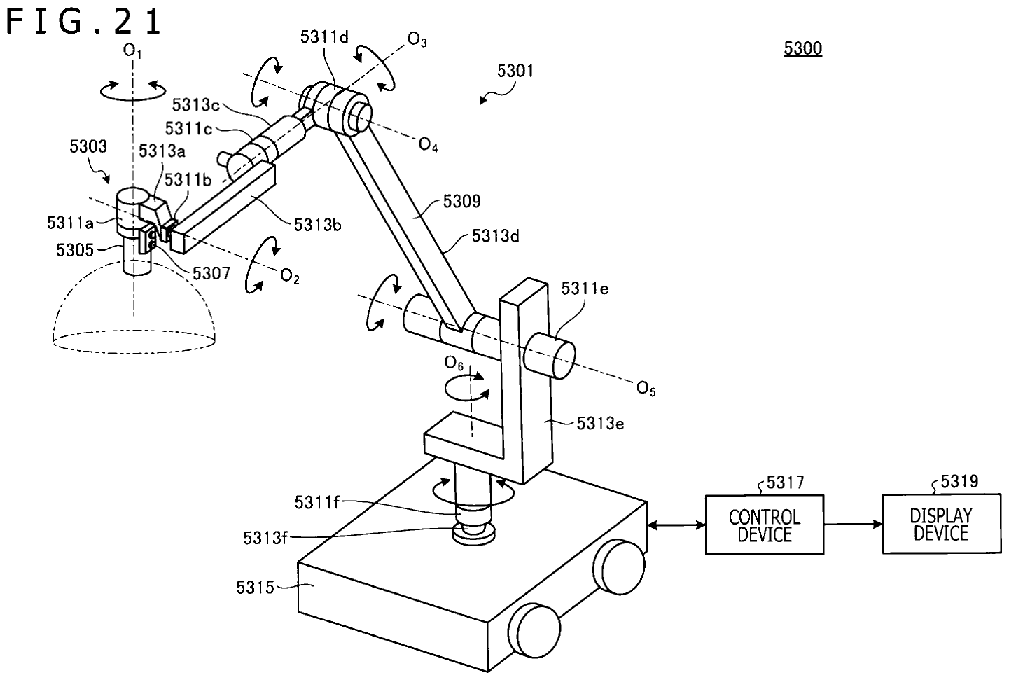

FIG. 21 is a view depicting an example of a schematic configuration of a microscopic surgery system 5300 to which techniques according to the present disclosure can be applied. Referring to FIG. 21, the microscopic surgery system 5300 is configured from a microscope device 5301 (the microscope device 5301 is an example of a medical observation apparatus), a control device 5317, and a display device 5319. It is to be noted that in the following description about the microscopic surgery system 5300, the term “user” means any medical staff, such as an operator or assistant, who uses the microscopic surgery system 5300.

The microscope device 5301 includes a microscope portion 5303 for observing an observation target (an operative field of a patient) under magnification, an arm portion 5309 supporting at a distal end thereof the microscope portion 5303, and a base portion 5315 supporting the arm portion 5309 at a proximal end thereof.

The microscope portion 5303 is configured from a substantially cylindrical barrel portion 5305 (also called “scope”), an imaging portion (not depicted) disposed inside the barrel portion 5305, a light source device (not depicted) configured to illuminate normal light or special light to an operative field, and an operating portion 5307 disposed on a region of a part of an outer circumference of the barrel portion 5305. The microscope portion 5303 is an electronic imaging microscope portion (so-called video microscope portion) that electronically captures an image by an imaging portion.

Over the plane of an opening in a lower end of the barrel portion 5305, a cover glass is disposed to protect the imaging portion inside. Light from an observation target (hereinafter also called “observed light”) passes through the cover glass, and enters the imaging portion inside the barrel portion 5305. It is to be noted that a light source including, for example, an LED (Light Emitting Diode) may be disposed inside the barrel portion 5305, and upon imaging, light may be illuminated from the light source to the observation target through the cover glass.

The imaging portion is configured from an optical system and an imaging device. The optical system condenses observed light, and the imaging device receives the observed light condensed by the optical system. The optical system is configured from a combination of a plurality of lenses including a zoom lens and a focus lens, and its optical characteristics are designed so that the observed light is focused on a light-receiving surface of the imaging device. The imaging device receives and photoelectrically converts the observed light, so that signals corresponding to the observed light, in other words, image signals corresponding to an observed image are generated. As the imaging device, one having a Bayer array to enable capture of a color image is used, for example. The imaging device may be one of various known imaging devices such as CMOS (Complementary Metal Oxide Semiconductor) image sensors and CCD (Charge Coupled Device) image sensors. The image signals generated by the imaging device are transmitted as RAW data to the control device 5317. Here, the transmission of the image signals may be suitably performed by optical communication. At an operation site, an operator performs surgery while observing the state of an affected area based on captured images. For safer and more reliable surgery, it is hence required to display a movie image of an operative field in as real time as possible. The transmission of image signals by optical communication enables to display a captured image with low latency.

It is to be noted that the imaging portion may also include a drive mechanism to cause movements of the zoom lens and focus lens along an optical axis in its optical system. By moving the zoom lens and focus lens with the drive mechanism as needed, the magnification of a captured image and the focal length during capturing can be adjusted. In addition, the imaging portion may also be mounted with various functions that can be generally included in electronically imaging microscope portions such as AE (Auto Exposure) function and AF (Auto Focus) function.

Further, the imaging portion may also be configured as a so-called single-plate imaging portion having a single imaging device, or may also be configured as a so-called multiplate imaging portion having a plurality of imaging devices. In a case where the imaging portion is configured as a multiplate imaging portion, a color image may be acquired, for example, by generating image signals corresponding to RGB, respectively, from respective imaging devices and combining the image signals. As an alternative, the imaging portion may also be configured so that a pair of imaging devices is included to acquire image signals for the right eye and left eye, respectively, and to enable stereovision (3D display). Performance of 3D display allows the operator to more precisely grasp the depth of a living tissue in an operative field. It is to be noted that, if the imaging portion is configured as a multiplate imaging portion, a plurality of optical systems can be also disposed corresponding to respective imaging devices.

The operating portion 5307 is configured, for example, by a four-directional lever or switch or the like, and is input means configured to receive an operational input by a user. Via the operating portion 5307, the user can input, for example, an instruction to the effect that the magnification of an observed image and the focal length to the observation target shall be changed. By moving the zoom lens and focus lens as needed via the drive mechanism of the imaging portion according to the instruction, the magnification and focal length can be adjusted. Via the operating portion 5307, the user can also input, for example, an instruction to the effect that operation mode (all free mode or fixed mode to be described subsequently herein) of the arm portion 5309 shall be switched. Further, if the user intends to move the microscope portion 5303, the user is expected to move the microscope portion 5303 in a state in which the user is grasping and holding the barrel portion 5305. Therefore, the operating portion 5307 is preferably disposed at a position where the user can easily operate the operating portion 5307 by fingers with the barrel portion 5305 grasped so that the operating portion 5307 can be operated even while the user is moving the barrel portion 5305.

The arm portion 5309 is configured with a plurality of links (first link 5313a to sixth link 53130 being connected rotatably relative to each other via a plurality of joint portions (first joint portion 5311a to sixth joint portion 5311f).

The first joint portion 5311a has a substantially columnar shape, and supports at a distal end (lower end) thereof an upper end of the barrel portion 5305 of the microscope portion 5303 rotatably about a rotational axis (first axis O₁) that is parallel to a central axis of the barrel portion 5305. Here, the first joint portion 5311a can be configured so that the first axis O₁ coincides with an optical axis of the imaging portion of the microscope portion 5303. As a consequence, rotation of the microscope portion 5303 about the first axis O₁ can change the field of vision so that a captured image is rotated.

The first link 5313a fixedly supports at a distal end thereof the first joint portion 5311a. Specifically, the first link 5313a is a rod-shaped member having a substantially L-shape, and is connected to the first joint portion 5311a so that its one arm on the side of a distal end thereof extends in a direction orthogonal to the first axis O₁ and is at an end portion thereof in contact with an upper end portion of an outer circumference of the first joint portion 5311a. The second joint portion 5311b is connected to an end portion of the other arm of the substantially L-shaped first link 5313a, the other arm being on the side of a proximal end of the first link 5313a.

The second joint portion 5311b has a substantially columnar shape, and supports at a distal end thereof the proximal end of the first link 5313a rotatably about a rotational axis (second axis O₂) that is orthogonal to the first axis O₁. The second link 5313b is fixedly connected at a distal end thereof to a proximal end of the second joint portion 5311b.

The second link 5313b is a rod-shaped member having a substantially L-shape. One arm on the side of the distal end of the second link 5313b extends in a direction orthogonal to the second axis O₂, and is fixedly connected at an end portion thereof to a proximal end of the second joint portion 5311b. The third joint portion 5311c is connected to the other arm of the substantially L-shaped second link 5313b, the other arm being on the side of a proximal end of the second link 5313b.

The third joint portion 5311c has a substantially columnar shape, and supports at a distal end thereof the proximal end of the second link 5313b rotatably about a rotational axis (third axis O₃) that is orthogonal to each of the first axis O₁ and second axis O₂. The third link 5313c is fixedly connected at a distal end thereof to the proximal end of the third joint portion 5311c. Rotation of a configuration on a distal end, the configuration including the microscope portion 5303, about the second axis O₂ and third axis O₃ can move the microscope portion 5303 so that the position of the microscope portion 5303 is changed in a horizontal plane. In other words, the field of vision for an image to be captured can be moved in a plane by controlling the rotation about the second axis O₂ and third axis O₃.

The third link 5313c is configured to have a substantially columnar shape on the side of the distal end thereof, and the third joint portion 5311c is fixedly connected at the proximal end thereof to a distal end of the columnar shape so that the third link 5313c and third joint portion 5311c both have substantially the same central axis. The third link 5313c has a prismatic shape on the side of the proximal end thereof, and the fourth joint portion 5311d is connected to an end portion of the third link 5313c.

The fourth joint portion 5311d has a substantially columnar shape, and supports at a distal end thereof the proximal end of the third link 5313c rotatably about a rotational axis (fourth axis O₄) that is orthogonal to the third axis O₃. The fourth link 5313d is fixedly connected at a distal end thereof to a proximal end of the fourth joint portion 5311d.

The fourth link 5313d is a rod-shaped member extending substantially linearly, extends so as to be orthogonal to the fourth axis O₄, and is fixedly connected to the fourth joint portion 5311d so that the fourth link 5313d is in contact at an end portion of the distal end thereof with a side wall of the substantially columnar shape of the fourth joint portion 5311d. The fifth joint portion 5311e is connected to a proximal end of the fourth link 5313d.

The fifth joint portion 5311e has a substantially columnar shape, and supports on the side of a distal end thereof the proximal end of the fourth link 5313d rotatably about a rotational axis (fifth axis O₅) that is parallel to the fourth axis O₄. The fifth link 5313e is fixedly connected at a distal end thereof to the proximal end of the fifth joint portion 5311e. The fourth axis O₄ and fifth axis O₅ are rotational axes that enable to move the microscope portion 5303 in an up-and-down direction. Rotation of a configuration on the side of a distal end, the configuration including the microscope portion 5303, about the fourth axis O₄ and fifth axis O₅ can adjust the height of the microscope portion 5303, in other words, the distance between the microscope portion 5303 and an observation target.

The fifth link 5313e is configured from a combination of a first member and a second member. The first member has a substantially L-shape in which one of arms thereof extends in a vertical direction and the other arm extends in a horizontal direction. The second member has a rod-shape and extends vertically downwardly from a horizontally-extending part of the first member. The fifth joint portion 5311e is fixedly connected at the proximal end thereof to a vicinity of an upper end of a vertically-extending part of the first member of the fifth link 5313e. The sixth joint portion 5311f is connected to a proximal end (lower end) of the second member of the fifth link 5313e.

The sixth joint portion 5311f has a substantially columnar shape, and supports on the side of a distal end thereof the proximal end of the fifth link 5313e rotatably about a rotational axis (sixth axis O₆) that is parallel to the vertical direction. The sixth link 5313f is fixedly connected at a distal end thereof to the proximal end of the sixth joint portion 5311f.

The sixth link 5313f is a rod-shaped member extending in the vertical direction, and is fixedly connected at a proximal end thereof to an upper surface of the base portion 5315.

The first joint portion 5311a to the sixth joint portion 5311f each have a rotatable range suitably set so that the microscope portion 5303 can move as desired. As a consequence, at the arm portion 5309 having the above-described configuration, movement in six degrees of freedom in total, including three translational degrees of freedom and three rotational degrees of freedom, can be realized for the movement of the microscope portion 5303. By configuring the arm portion 5309 so that six degrees of freedom are realized concerning movement of the microscope portion 5303 as described above, the position and posture of the microscope portion 5303 can be freely controlled within the movable range of the arm portion 5309. Accordingly, an operative field can be observed from every angle, so that smoother surgery can be performed.

It is to be noted that the configuration of the arm portion 5309 depicted in figure is merely illustrative, and the number and shapes (lengths) of links and the number, disposed positions, directions of rotational axes and the like of joint portions, which make up the arm portion 5309, may be suitably designed so that desired degrees of freedom can be realized. To freely move the microscope portion 5303 as described above, for example, it is preferred to configure the arm portion 5309 so that it has six degrees of freedom. However, the arm portion 5309 may be configured to have still greater degrees of freedom (in other words, redundant degrees of freedom). If redundant degrees of freedom exist, the posture of the arm portion 5309 can be changed with the microscope portion 5303 being fixed in position and posture. It is hence possible to realize control more convenient to an operator such as, for example, to control the posture of the arm portion 5309 so that the arm portion 5309 does not interfere with the field of vision of the operator who is watching the display device 5319.

Now, actuators can be disposed in the first joint portion 5311a to the sixth joint portion 5311f, respectively. On each actuator, a drive mechanism such as an electric motor, an encoder configured to detect the angle of rotation at the corresponding joint portion, and the like can be mounted. The posture of the arm portion 5309, in other words, the position and posture of the microscope portion 5303 can be controlled through suitable control of the driving of the respective actuators, which are disposed in the first joint portion 5311a to the sixth joint portion 5311f, by the control device 5317. Specifically, the control device 5317 can grasp the current posture of the arm portion 5309 and the current position and posture of the microscope portion 5303 based on information regarding the rotation angles of the respective joint portions as detected by the encoder. Using the grasped information, the control device 5317 calculates control values (for example, rotation angles or torques to be produced) for the respective joint portions so that movement of the microscope portion 5303 according to an operational input from the user can be realized, and drives the drive mechanisms of the respective joint portions according to the control values. It is to be noted that in the above-described control, no limitation is imposed on the control method of the arm portion 5309 by the control device 5317 and various known control methods such as force control and position control may each be applied.

By suitably performing, by the operator, an operational input via an undepicted input device, for example, the driving of the arm portion 5309 is suitably controlled via the control device 5317 according to the operational input to control the position and posture of the microscope portion 5303. By the control, the microscope portion 5303 can be moved from any position to a desired position, and can then be fixedly supported at the position after the movement. As the input device, one that is operable even if the operator has a surgical instrument in hand, such as a foot switch, may preferably be applied in view of the operator's convenience. As an alternative, an operational input may also be performed without contact based on the detection of a gesture or sightline with a wearable device or a camera arranged in the operating room. As a consequence, it is possible even for a user who belongs to a clean area to operate with a higher degree of freedom equipment which belongs to an unclean area. As a further alternative, the arm portion 5309 may also be operated by a so-called master-slave method. In this case, the arm portion 5309 can be remote controlled by the user via an input device installed at a place remote from the operating room.

If force control is applied, on the other hand, a so-called power assist control may be performed, in which an external force from the user is received, and the actuators of the first joint portion 5311a to the sixth joint portion 5311f are driven so that the arm portion 5309 smoothly moves according to the external force. As a consequence, the user can move the microscope portion 5303 with a relatively light force upon directly moving the position of the microscope portion 5303 while grasping the microscope portion 5303. The microscope portion 5303 can hence be moved more intuitively with simpler operation, so that the user's convenience can be improved.

Further, the arm portion 5309 may be controlled in its driving so that it moves in a pivotal motion. The term “pivotal motion” as used herein means a motion which causes the microscope portion 5303 to move so that the optical axis of the microscope portion 5303 is maintained directed toward a predetermined point (hereinafter called “the pivot point”) on the space. According to the pivot motion, the same position of observation can be observed from various directions, and therefore more detailed observation of the affected area is possible. It is to be noted that, if the microscope portion 5303 is configured to be incapable of being adjusted in focal length, the pivotal motion may preferably be performed with the distance between the microscope portion 5303 and the pivot point being maintained fixed. In this case, it is only required to adjust beforehand the distance between the microscope portion 5303 and the pivot point to the fixed focal length of the microscope portion 5303. As a consequence, the microscope portion 5303 is to move on a hemispherical surface (depicted schematically in FIG. 21) that has a radius corresponding to the focal length about the pivot point as a center, and is to enable the acquisition of a clear captured image even if the direction of observation is changed. If the microscope portion 5303 is configured to be capable of being adjusted in focal length, on the other hand, the pivotal motion may be performed with the length between the microscope portion 5303 and the pivot point being maintained variable. In this case, the control device 5317, for example, may calculate the distance between the microscope portion 5303 and the pivot point based on information regarding the rotation angles at the respective joint portions as detected by the associated encoders, and may automatically adjust the focal length of the microscope portion 5303 based on the calculation results. As an alternative, if the microscope portion 5303 has an AF function, the adjustment of the focal length may be automatically performed by the AF function every time the distance between the microscope portion 5303 and the pivot point changes by a pivotal motion.

In addition, the first joint portion 5311a to the sixth joint portions 5311f may include brakes to restrain their rotation, respectively. Operation of the brakes can be controlled by the control device 5317. If it is desired to fix the position and posture of the microscope portion 5303, for example, the control device 5317 actuates the brakes in the respective joint portions. As a consequence, the position of the arm portion 5309, in other words, the position and posture of the microscope portion 5303 can be fixed without driving the actuators, and therefore power consumption can be reduced. If it is desired to change the position and posture of the microscope portion 5303, it is only required for the control device 5317 to release the brakes at the respective joint portions and to drive the actuators according to a predetermined control method.

Such operation of the brakes can be performed in response to the above-described operational input by the user via the operating portion 5307. If desired to change the position and posture of the microscope portion 5303, the user operates the operating portion 5307 to release the brakes at the respective joint portions. As a consequence, the operation mode of the arm portion 5309 is changed to a mode (all free mode) that allows to freely perform rotation at the respective joint portions. If desired to fix the position and posture of the microscope portion 5303, on the other hand, the user operates the operating portion 5307 to actuate the brakes at the respective joint portions. As a consequence, the operation mode of the arm portion 5309 is changed to a mode (fixed mode) in which rotation is restrained at the respective joint portions.

By controlling the operation of the microscope device 5301 and display device 5319, the control device 5317 comprehensively controls the operation of the microscopic surgery system 5300. For example, the control device 5317 controls the driving of the arm portion 5309 by operating the actuators of the first joint portion 5311a to the sixth joint portion 5311f according to a predetermined control method. As another example, the control device 5317 changes the operation mode of the arm portion 5309 by controlling the operation of the brakes of the first joint portion 5311a to the sixth joint portion 5311f. As a further example, the control device 5317 generates image data for display purpose by applying various signal processing to image signals acquired by the imaging portion of the microscope portion 5303 of the microscope device 5301, and then causes the display device 5319 to display the image data. In the signal processing, a variety of known signal processing such as, for example, development processing (demosaicing processing), image quality enhancement processing (band enhancement processing, super resolution processing, NR (Noise reduction) processing, and/or image stabilization processing), and/or magnification processing (in other words, electronic zooming processing) may be performed.

Communications between the control device 5317 and the microscope portion 5303 and communications between the control device 5317 and the first joint portion 5311a to the sixth joint portion 5311f may be wired communications or wireless communications. In the case of wired communications, communications by electrical signals may be performed or optical communications may be performed. In this case, transmission cables for use in wired communications can be configured as electrical signal cables, optical fibers, or composite cables thereof, depending on its communication method. In the case of wireless communications, on the other hand, it is no longer needed to lay transmission cables in the operating room. Accordingly, it is possible to eliminate a situation in which movements of medical staff in the operating room are interfered by the transmission cables.

The control device 5317 can be a microcomputer, a control board or the like, with processors such as a CPU (Central Processing Unit) and GPU (Graphic Processing Unit) or processors and a storage device such as a memory being mounted together. The processors of the control device 5317 operate according to predetermined programs, whereby the above-described various functions can be realized. It is to be noted that the control device 5317 is disposed as a device discrete from the microscope device 5301 in the example depicted in the figure but the control device 5317 may be arranged inside the base portion 5315 of the microscope device 5301 and may be configured integrally with the microscope device 5301. As an alternative, the control device 5317 may be configured from a plurality of devices. For example, microcomputers, control boards and the like may be arranged in the microscope portion 5303 and the first joint portion 5311a to the sixth joint portions 5311f of the arm portion 5309, respectively, and are connected for mutual communications, whereby functions similar to those of the control device 5317 may be realized.

The display device 5319 is placed inside the operating room, and under control from the control device 5317, displays an image corresponding to image data as generated by the control device 5317. In other words, an image of an operative field as captured by the microscope portion 5303 is displayed on the display device 5319. It is to be noted that the display device 5319 may display, instead of or together with the image of the operative field, various kinds of information regarding the surgery such as, for example, the patient's physical information and the operative method of the surgery. In this case, the display on the display device 5319 may be switched as needed by the user's operation, or a plurality of display devices 5319 may be arranged and the image of the operative field and the various kinds of information regarding the surgery may be displayed on the display devices 5319, respectively. It is to be noted that as the display device 5319, desired one or more of various known display devices, such as a liquid crystal display devices or an EL (Electro Luminescence) display devices, may be applied.

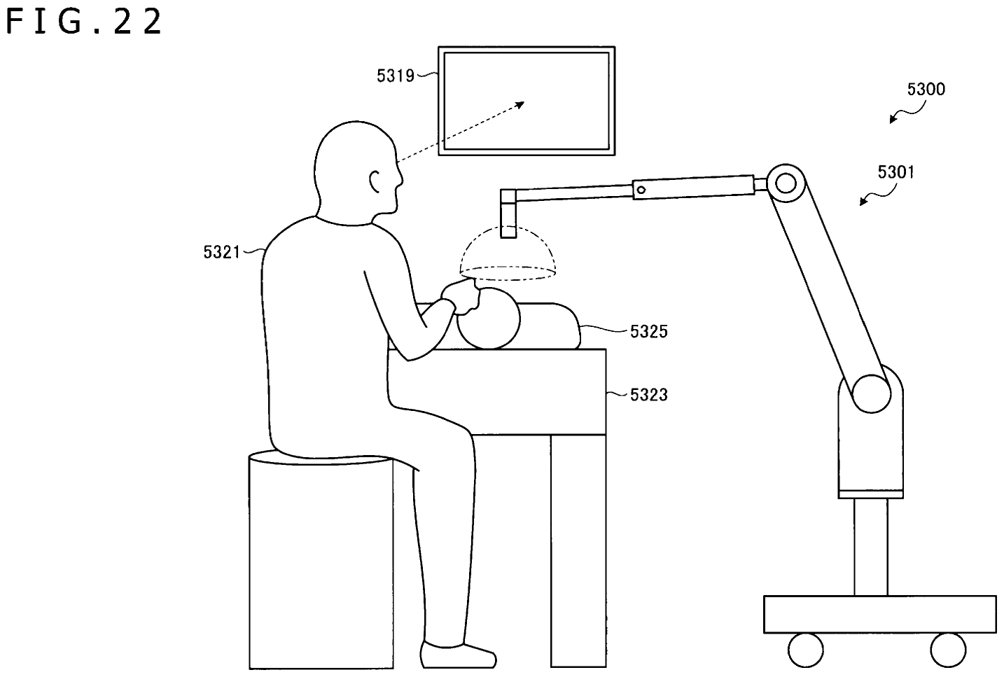

FIG. 22 is a view illustrating how surgery is being performed using the microscopic surgery system 5300 depicted in FIG. 21. FIG. 22 schematically illustrates how an operator 5321 is performing surgery on a patient 5325 on a patient bed 5323 by using the microscopic surgery system 5300. It is to be noted that in FIG. 22, illustration of the control device 5317 out of the configuration of the microscopic surgery system 5300 is omitted for the sake of simplicity and the microscope device 5301 is illustrated in a simplified form.

As illustrated in FIG. 22, during the surgery, the microscopic surgery system 5300 is used, and an image of an operative field as captured by the microscope device 5301 is displayed under magnification on the display device 5319 disposed on a wall surface of an operating room. The display device 5319 is disposed at a position opposite the operator 5321, and the operator 5321 performs various treatment, such as, for example, resection of the affected area, to the operative field while observing the conditions of the operative field based on the image displayed on the display device 5319.

In the embodiments described above, the performance of superimposition of an image on an estimated region was described. Predetermined image processing, for example, image enhancement processing such as linear enhancement processing and color enhancement, binarization processing and/or sharpness enhancement processing may be applied based on the estimated region. Further, image processing may be applied not only to the estimated region, but also based on the estimated region. For example, instead of applying image processing to the estimated region itself, superimposed image processing may be applied to a region based on the estimated region, such as surrounding a little outside the estimated region with a dotted line based on the estimated region.

The description has been made hereinabove about an example of the microscopic surgery system 5300 to which the techniques relating to the present disclosure can be applied. Although the microscopic surgery system 5300 has herein been described as an illustrative example, systems to which the techniques according to the present disclosure can be applied are not limited to such an example. For example, the microscope device 5301 can also function as a support arm device that supports at a distal end thereof another medical observation apparatus or another surgical instrument instead of the microscope portion 5303. As such another medical observation apparatus, an endoscope can be applied, for example. As such another surgical instrument, on the other hand, forceps, tweezers, an insufflator tube for insufflation, an energy treatment instrument for performing incision of a tissue or sealing of blood vessels by cauterization, or the like can be applied. By supporting such an observation device or a surgical instrument with the support arm device, medical staff can more stably fix its position compared with manually supporting the same, and at the same time, can alleviate the load on the medical staff. The techniques according to the present disclosure may be applied to a support arm device that supports such a configuration other than the microscope portion.

It is to be noted that the advantageous effects described herein are merely illustrative and non-limiting, and other advantageous effects may also be brought about.

It is also to be noted that the present techniques can be configured as will be described hereinafter.

(1)
A medical observation system including:
a generating section that generates three-dimensional information regarding an operative field;
a setting section that sets, based on a special light image captured by a medical observation apparatus during illumination of special light having a predetermined wavelength bandwidth, an interested region in the special light image;
a calculating section that estimates, from the three-dimensional information, an estimated region corresponding to a physical position of the interested region in a normal light image captured by the medical observation apparatus during illumination of normal light having a wavelength bandwidth different from the predetermined wavelength bandwidth; and
an image processing section that applies predetermined image processing to the estimated region in the normal light image.

(2)
The medical observation system as described above in (1), in which
the generating section generates the three-dimensional information from at least two pieces of the normal light images of the operative field as captured at different angles by the medical observation apparatus.

(3)
The medical observation system as described above in (2), in which
the generating section generates the three-dimensional information by matching feature points in the at least two pieces of the normal light images.

(4)
The medical observation system as described above in (3), in which
the three-dimensional information includes at least map information representing three-dimensional coordinates of the operative field, position information regarding the medical observation apparatus, and posture information regarding the medical observation apparatus.

(5)
The medical observation system as described above in (4), in which
the calculating section calculates interested coordinates which correspond to a physical position of the interested region at the three-dimensional coordinates by using the map information, and based on the map information, position information and posture information, estimates as the estimated region a region corresponding to the interested coordinates in the normal light image.

(6)
The medical observation system as described above in (5), in which
the image processing section performs the image processing by superimposing, on the estimated region in the normal light image, annotation information which includes information representing features of the special light image.

(7)
The medical observation system as described above in (5), in which
the image processing section applies image enhancement processing to the estimated region, the image enhancement processing being different from that to be applied to a region outside the estimated region.

(8)
The medical observation system as described above in any one of (1) to (7), in which
the setting section receives an instruction to set the interested region.

(9)
The medical observation system as described above in (8), in which
the setting section receives an input instructing a timing at which the interested region is to be set.

(10)
The medical observation system as described above in (8), in which the setting section receives an input instructing a target to be selected from one or more feature regions in the special light image and to be set as the interested region.

(11)
The medical observation system as described above in any one of (1) to (10), in which
the image processing section applies the image processing which add information regarding the interested region to the estimated region in the normal light image.

(12)
The medical observation system as described above in (11), in which
the image processing section performs the image processing to add information that represents whether or not the estimated region is at a preset distance from a contour of the interested region.

(13)
The medical observation system as described above in any one of (1) to (12), in which
the image processing section applies the image processing to the estimated region based on a feature value of a feature region in the special light image.

(14)
The medical observation system as described above in (13), in which
the setting section sets, as the interested region, a fluorescence-emitting region in the special light image, and
the image processing section applies the image processing to the estimated region based on an intensity of fluorescence of the interested region.

(15)
The medical observation system as described above in (13), in which
the setting section sets the interested region based on a state of blood in the operative field,
the calculating section estimates the estimated region corresponding to the physical position of the interested region, and
the image processing section applies the image processing which represents the state of the blood to the estimated region in the normal light image.

(16)
The medical observation system as described above in any one of (1) to (15), in which
the image processing section updates the image processing to be applied to the estimated region.

(17)
The medical observation system as described above in (3), in which
the generating section detects the feature points from outsides of the interested regions.

(18)
The medical observation system as described above in (17), in which
the generating section detects the feature points from regions other than regions in which a predetermined tool included in the normal light images has been detected.

(19)
The medical observation system as described above in any one of (1) to (18), including:
a light source device illuminates the normal light or the special light onto the operative field via a scope inserted to the operative field; and
a signal processing apparatus processes signals from an imaging device which receives light guided from the scope and transmits the processed signals to a display device, in which
the medical observation apparatus has a casing to which the scope is connectable, and the imaging device disposed in the casing, and
the signal processing apparatus has a circuit realizes functions of at least the generating section, the setting section, the calculating section and the image processing section.

(20)
The medical observation system as described above in any one of (1) to (19), in which
the generating section acquires distance information regarding the operative field, and generates the three-dimensional information by matching feature points in the distance information.

(21)
A signal processing apparatus including:
a generating section that generates three-dimensional information regarding an operative field;
a setting section that sets, based on a special light image captured by a medical observation apparatus during illumination of special light having a predetermined wavelength bandwidth, an interested region in the special light image;
a calculating section that estimates, from the three-dimensional information, an estimated region corresponding to a physical position of the interested region in a normal light image captured by the medical observation apparatus during illumination of normal light having a wavelength bandwidth different from the predetermined wavelength bandwidth; and
an image processing section that applies predetermined image processing to the estimated region in the normal light image.

(22)
A medical observation method including:
generating three-dimensional information regarding an operative field;
setting, based on a special light image captured by a medical observation apparatus during illumination of special light having a predetermined wavelength bandwidth, an interested region in the special light image;
estimating, from the three-dimensional information, an estimated region corresponding to a physical position of the interested region in a normal light image captured by the medical observation apparatus during illumination of normal light having a wavelength bandwidth different from the predetermined wavelength bandwidth; and
applying predetermined image processing to the estimated region in the normal light image.
It is also to be noted that the present techniques can be configured as will be described hereinafter.

(1)
A medical observation system comprising:

circuitry configured to:
obtain a first surgical image captured by a medical imaging apparatus during illumination in a first wavelength band and a second surgical image captured by the medical imaging apparatus during illumination in a second wavelength band different from the first wavelength band,
generate three-dimensional information regarding an operative field,
obtain information of an interested region in the first surgical image,
calculate, based on the three-dimensional information, an estimated region in the second surgical image corresponding to a physical position of the interested region, and
output the second surgical image processed a predetermined image processing on the estimated region.

(2)
The medical observation system as described above in (1), in which the circuitry is configured to generate the three-dimensional information based on at least two pieces of the second surgical images of the operative field as captured at different angles by the medical imaging apparatus.

(3)
The medical observation system as described above in (1) or (2), in which the circuitry is configured to generate the three-dimensional information by matching feature points in the at least two pieces of the second surgical images.

(4)
The medical observation system as described above in any one of (1) to (3), in which the circuitry is configured to generate the three-dimensional information includes at least map information representing three-dimensional coordinates of the operative field, position information regarding the medical imaging apparatus, and posture information regarding the medical imaging apparatus.

(5)
The medical observation system as described above in any one of (1) to (4), in which the circuitry is configured to generate the three-dimensional information by Simultaneous Localization and Mapping processing based on the second surgical image.

(6)
The medical observation system as described above in (5), in which the circuitry is configured to calculate the estimated region in the second surgical image corresponding to the physical position of the interested region by calculating interested coordinates corresponding to the physical position of the interested region at the map information based on the map information position information and posture information.

(7)
The medical observation system as described above in any one of (1) to (6), in which the circuitry is configured to perform the predetermined image processing by superimposing on the estimated region in the second surgical image, annotation information which includes information representing features of first surgical image.

(8)
The medical observation system as described above in any one of (1) to (7), in which the circuitry is configured to apply image enhancement processing to the second surgical image; and
the image enhancement processing differs in processing parameter between the estimated region and outside of the estimated region.

(9)
The medical observation system as described above in any one of (1) to (8), in which the illumination in the first wavelength band is an infrared light, and the illumination in the second wavelength band is a white light.

(10)
The medical observation system as described above in any one of (1) to (9), in which the circuitry is configured to receive an input instructing a target to be selected from one or more feature regions in the first surgical image and to be set as the interested region.

(11)
The medical observation system as described above in any one of (1) to (10), in which the circuitry is configured to apply the image processing to add information regarding the interested region on the estimated region in the second surgical image.

(12)
The medical observation system as described above in any one of (1) to (11), in which the circuitry is configured to perform the image processing to add information that represents whether or not the estimated region is at a preset distance from a contour of the interested region.

(13)
The medical observation system as described above in any one of (1) to (12), in which the circuitry is configured to apply the image processing to the estimated region based on a feature value of a feature region in the first surgical image.

(14)
The medical observation system as described above in any one of (1) to (13), in which the circuitry is configured to set, as the interested region, a fluorescence-emitting region in the first surgical image and apply the image processing to the estimated region based on an intensity of fluorescence of the interested region.

(15)
The medical observation system as described above in any one of (1) to (14), in which the circuitry is configured to:
set the interested region based on a state of blood in the operative field,
estimate the estimated region corresponding to the physical position of the interested region, and
apply the image processing which represents the state of the blood to the estimated region in the second surgical image.

(16)
The medical observation system as described above in any one of (1) to (15), in which the circuitry is configured to detect a feature points from outside of the interested region.

(17)
The medical observation system as described above in any one of (16), in which the circuitry is configured to detect the feature points from regions other than regions in which a predetermined tool included in the second surgical images has been detected.

(18)
The medical observation system as described above in any one of (1) to (17), further comprising:
a light source device including a special light source that illuminate in the first wavelength band and a normal light source that illuminate in the second wavelength band;
an endoscope including the medical imaging apparatus connectable a scope; and
a medical processing apparatus including the circuitry;

wherein, the circuitry is configured to obtain the first surgical image captured by the endoscope while the special light source illuminates the operative field and the second surgical image captured by the endoscope while the normal light source illuminates the operative field.

(19)
A signal processing apparatus comprising:

circuitry configured to:
obtain a first surgical image captured by a medical imaging apparatus during illumination in a first wavelength band and a second surgical image captured by the medical imaging apparatus during illumination in a second wavelength band different from the first wavelength band,
generate three-dimensional information regarding an operative field,
obtain information of an interested region in the first surgical image,
output, based on the three-dimensional information, an estimated region processed a predetermined image processing in the second surgical image corresponding to a physical position of the interested region.

(20)
A medical observation method by a medical observation apparatus including circuitry, including:
obtaining a first surgical image captured by a medical imaging apparatus during illumination in a first wavelength band and a second surgical image captured by the medical imaging apparatus during illumination in a second wavelength band different from the first wavelength band,
generating three-dimensional information regarding an operative field,
obtaining information of an interested region in the first surgical image,
calculating, based on the three-dimensional information, an estimated region in the second surgical image corresponding to a physical position of the interested region,
outputting the second surgical image processed a predetermined image processing on the estimated region.

### REFERENCE SIGNS LIST

5000 Endoscopic surgery system
5001 Endoscope
1000, 1000a, 1000b, 1000c, 1000d, 1000e, 1000f, 1000g, 1000h, 1000i, 1000j, 1000k Medical observation system
2000, 2000a, 2000b, 2000c, 2000d, 2000e, 2000f, 2000g, 2000h, 2000i, 2000j, 2000k Imaging apparatus
100, 101, 110 Imaging device
120 Imaging and phase-difference sensor
200, 201 Special light imaging device
300 Depth sensor
5039, 5039d, 5039e, 5039f, 5039g, 5039j, 5039k CCU
11 Normal light development processing section
12 Special light development processing section
21 Three-dimensional information generating section
22 Map generation section
23 Self-position estimation section
24 Three-dimensional information storage section
31 Interested region setting section
32 Estimated region calculating section
41 Image processing section
51 Display control section
61 AE detection section
62 AE control section
63 Light source control section
71 Depth information generating section
81 Tracking processing section
5300 Microscopic surgery system
5301 Microscope device
G1 Annotation information
G11 Interested region information
G12 Area size information
G13 Boundary line information
G14 Distance-to-boundary information
G2 Provisional annotation information
G3 Specifying line
R1 Interested region

1. A medical observation system comprising:
circuitry configured to:
obtain a first surgical image captured by a medical imaging apparatus during illumination in a first wavelength band and a second surgical image captured by the medical imaging apparatus during illumination in a second wavelength band different from the first wavelength band,
generate three-dimensional information regarding an operative field,
obtain information of an interested region in the first surgical image,
calculate, based on the three-dimensional information, an estimated region in the second surgical image corresponding to a physical position of the interested region, and
output the second surgical image processed a predetermined image processing on the estimated region.

2. The medical observation system according to claim 1, wherein the circuitry is configured to generate the three-dimensional information based on at least two pieces of the second surgical images of the operative field as captured at different angles by the medical imaging apparatus.

3. The medical observation system according to claim 2, wherein the circuitry is configured to generate the three-dimensional information by matching feature points in the at least two pieces of the second surgical images.

4. The medical observation system according to claim 3, wherein the circuitry is configured to generate the three-dimensional information includes at least map information representing three-dimensional coordinates of the operative field, position information regarding the medical imaging apparatus, and posture information regarding the medical imaging apparatus.

5. The medical observation system according to claim 4, wherein the circuitry is configured to generate the three-dimensional information by Simultaneous Localization and Mapping processing based on the second surgical image.

6. The medical observation system according to claim 5, wherein the circuitry is configured to calculate the estimated region in the second surgical image corresponding to the physical position of the interested region by calculating interested coordinates corresponding to the physical position of the interested region at the map information based on the map information position information and posture information.

7. The medical observation system according to claim 6, wherein the circuitry is configured to perform the predetermined image processing by superimposing on the estimated region in the second surgical image, annotation information which includes information representing features of first surgical image.

8. The medical observation system according to claim 6, wherein the circuitry is configured to apply image enhancement processing to the second surgical image; and
the image enhancement processing differs in processing parameter between the estimated region and outside of the estimated region.

9. The medical observation system according to claim 1, wherein the illumination in the first wavelength band is an infrared light, and
the illumination in the second wavelength band is a white light.

10. The medical observation system according to claim 8, wherein the circuitry is configured to receive an input instructing a target to be selected from one or more feature regions in the first surgical image and to be set as the interested region.

11. The medical observation system according to claim 1, wherein the circuitry is configured to apply the image processing to add information regarding the interested region on the estimated region in the second surgical image.

12. The medical observation system according to claim 11, wherein the circuitry is configured to perform the image processing to add information that represents whether or not the estimated region is at a preset distance from a contour of the interested region.

13. The medical observation system according to claim 1, wherein the circuitry is configured to apply the image processing to the estimated region based on a feature value of a feature region in the first surgical image.

14. The medical observation system according to claim 13, wherein the circuitry is configured to set, as the interested region, a fluorescence-emitting region in the first surgical image and apply the image processing to the estimated region based on an intensity of fluorescence of the interested region.

15. The medical observation system according to claim 13, wherein the circuitry is configured to:
set the interested region based on a state of blood in the operative field,
estimate the estimated region corresponding to the physical position of the interested region, and
apply the image processing which represents the state of the blood to the estimated region in the second surgical image.

16. The medical observation system according to claim 1, wherein the circuitry is configured to detect a feature points from outside of the interested region.

17. The medical observation system according to claim 16, wherein the circuitry is configured to detect the feature points from regions other than regions in which a predetermined tool included in the second surgical images has been detected.

18. The medical observation system according to claim 1, further comprising:
a light source device including a special light source that illuminate in the first wavelength band and a normal light source that illuminate in the second wavelength band;
an endoscope including the medical imaging apparatus connectable a scope; and
a medical processing apparatus including the circuitry;
wherein, the circuitry is configured to obtain the first surgical image captured by the endoscope while the special light source illuminates the operative field and the second surgical image captured by the endoscope while the normal light source illuminates the operative field.

19. A signal processing apparatus comprising:
circuitry configured to:
obtain a first surgical image captured by a medical imaging apparatus during illumination in a first wavelength band and a second surgical image captured by the medical imaging apparatus during illumination in a second wavelength band different from the first wavelength band,
generate three-dimensional information regarding an operative field,
obtain information of an interested region in the first surgical image,
output, based on the three-dimensional information, an estimated region processed a predetermined image processing in the second surgical image corresponding to a physical position of the interested region.

20. A medical observation method by a medical observation apparatus including circuitry, including:
obtaining a first surgical image captured by a medical imaging apparatus during illumination in a first wavelength band and a second surgical image captured by the medical imaging apparatus during illumination in a second wavelength band different from the first wavelength band,
generating three-dimensional information regarding an operative field,
obtaining information of an interested region in the first surgical image,
calculating, based on the three-dimensional information, an estimated region in the second surgical image corresponding to a physical position of the interested region,
outputting the second surgical image processed a predetermined image processing on the estimated region.
